(19) United States
(12) Patent Application Publication
(10) Pub. No.: US 2026/0159887 A1
(43) Pub. Date: Jun. 11, 2026

(54) NON-INVASIVE DIRECT TRANSCRIPTOMIC PROFILING USING STOOL SAMPLES FOR DIAGNOSIS OF GASTROINTESTINAL DISEASES

(71) Applicant: COLORADO STATE UNIVERSITY RESEARCH FOUNDATION, Fort Collins, CO (US)

(72) Inventors: Steven Dow, Fort Collins, CO (US); Lyndah Chow, Fort Collins, CO (US); Alison Manchester, Fort Collins, CO (US)

(21) Appl. No.: 18/706,455

(22) PCT Filed: Nov. 1, 2022
(86) PCT No.: PCT/US2022/048614

§ 371 Date: May 1, 2024

Related U.S. Application Data
(60) Provisional application No. 63/274,380, filed on Nov. 1, 2021.
(60) Provisional application No. 63/290,849, filed on Dec. 17, 2021.

(51) Int. Cl. C12Q 1/6883; C12Q 1/6806

(52) U.S. Cl. CPC C12Q 1/6883; C12Q 1/6806; C12Q 2600/158

(57) ABSTRACT

Inflammatory bowel disease (IBD) in humans and dogs and other companion animals is characterized by infiltration of lymphocytes and macrophages into the mucosa and submucosa and clinical signs of gastrointestinal (GI) dysfunction (diarrhea, malabsorption, weight loss). Alteration of the gut environment and development of dysbiosis may allow the overgrowth of pathogenic bacteria and induction of intestinal injury and inflammation in IBD. What is needed are novel methods that allow for rapid diagnosis of IBD (and follow-up monitoring) and treatment of the disease. The present disclosure relates to methods for diagnosing and treating inflammatory bowel disease (IBD) or gastrointestinal lymphoma.

### CROSS-REFERENCE TO RELATED APPLICATIONS

This application claims the benefit of U.S. Provisional Application No. 63/274,380, filed Nov. 1, 2021, and U.S. Provisional Application No. 63/290,849, filed Dec. 17, 2021, which are expressly incorporated herein by reference in their entireties.

### FIELD

The present disclosure relates to methods for diagnosing and treating gastrointestinal disorders.

### BACKGROUND

Inflammatory bowel disease (IBD) in humans and dogs and other companion animals is characterized by infiltration of lymphocytes and macrophages into the mucosa and submucosa and clinical signs of gastrointestinal (GI) dysfunction (diarrhea, malabsorption, weight loss). Alteration of the gut environment and development of dysbiosis may allow the overgrowth of pathogenic bacteria and induction of intestinal injury and inflammation in IBD. Genetic and environmental factors are also associated with development of IBD in dogs and humans, independent of the role of the intestinal microbiome. Currently the diagnosis of IBD in humans and veterinary species is complicated, time consuming, costly, and often involves the use of endoscopy or colonoscopy procedures, all of which increases cost and risks. What is needed are novel methods that allow for rapid diagnosis of IBD (and follow-up monitoring) and treatment of the disease. Said methods, which would not require invasive GI biopsy procedures, represent an important advance in the diagnosis of IBD in humans and veterinary species (companion animals). The methods disclosed herein address these and other needs.

### SUMMARY

In some aspects, disclosed herein are method of measuring a level of a coding or non-coding nucleic acid (such as mRNA, microRNA, or DNA) in a stool sample, comprising:
a) obtaining a stool sample;
b) extracting the RNA (or DNA) from the stool sample; and
c) measuring the level of the coding or non-coding RNA or DNA.

In some embodiments, the stool sample of step a) is placed or stored in an RNA preservation solution.

In some embodiments, step b) further comprises:
adding the stool sample in a tube that comprises a lysis buffer, wherein the lysis buffer comprises β-mercaptoethanol;
mixing the stool sample with the lysis buffer; and
centrifuging the mixed sample thereby obtaining a first supernatant portion.

In some embodiments, the method of any preceding aspect further comprises adding a solution comprising phenol, chloroform, and isoamyl alcohol into the tube prior to adding the stool sample. In some embodiments, the method of any preceding aspect further comprises adding a solution comprising phenol into the tube prior to adding the stool sample.

In some embodiments, the method of any preceding aspect further comprises:
transferring the first supernatant portion into a tube;
mixing the first supernatant portion with an inhibitor removal solution in the tube; and
centrifuging the mixed sample thereby obtaining a second supernatant portion.

In some embodiments, the method of any preceding aspect further comprises
transferring the second supernatant portion into a tube;
mixing the second supernatant portion with a solution comprising binding salts and ethanol; and
passing the mixture through a binding membrane thereby binding the RNA (or DNA) onto the membrane.

In some embodiments, the method of any preceding aspect further comprises adding a DNase to the binding membrane. It should be understood and herein contemplated that the step of adding the DNase is to remove contaminating DNA, while in other applications the DNA would not be removed, and would be the primary analyte instead. In some embodiments, the method of any preceding aspect further comprises washing the binding membrane with a washing buffer comprising isopropanol or ethanol. In some embodiments, the method of any preceding aspect further comprises adding water to the binding membrane thereby eluting the RNA in the water.

In some embodiments, the method of any preceding aspect comprises measuring the level of the RNA with an RNA sequencing assay, a hybridization-based array, or a polymerase chain reaction (PCR)-based assay (e.g., reverse transcription PCR (RT-PCR) or real-time RT PCR). In some embodiments, the RNA is measured by NanoString analysis, real-time PCR, or Illumina or other high-throughput next generation sequencing analysis.

In some embodiments, the level of the RNA is measured by one or more primers or probes specific for RNAs encoded by genes selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567.

In some embodiments, the level of the RNA is measured by one or more primers or probes specific for RNAs encoded by genes selected from the group consisting of LOC489428, DLA-DQB1, IDO1, CCL25, BAX, IL12RB2, PSMB8, STAT2, CSF3R, NOD1, S100A4, and TRGC2.

In some embodiments, the level of the RNA is measured by one or more primers or probes specific for RNAs encoded by genes selected from the group consisting of IFNA7, TLR4, C7, CD96, TFEB, FCAR, LOC487977, BLNK, GSK38, and LOC1021567.

In some embodiments, the level of the RNA is measured by one or more primers or probes specific for RNAs encoded by genes selected from the group consisting of TNFRSF13C, CXCL10, SHMT2, CX3CL1, PPARGC1A, DMBT1, FYN, MAVS, CD163, CCL19, SIGIRR, CD84, TXNIP, TXNIP, PRKCD, IL17F, NRAS, C7, C5, CFD, CSF3, LOC483397, and MAPK1.

In some embodiments, the level of the RNA is measured by one or more primers or probes specific for RNAs encoded by genes selected from the group consisting of MSR1, ARG1, IL11RA, IL2RG, CTSS, RUNX1, CCR1, ADA, TIGIT, MR1, IL27, CRADD, PSMB8, and HLA-DRB3.

In some embodiments, the level of the RNA is measured by one or more primers or probes specific for RNAs encoded by genes selected from the group consisting of S100A8, IDO1, CXCL8, FOS, IL1RN, EGR1, CEBPB, EPCAM, S100A9, PLAUR, S100A12, DUSP1, CD74, NFKBIA, LCP1, C1QBP, TNFAIP3, DLA-DRA, DDX5, LGALS3, CCND2, MAP2K2, MEF2C, PSMB9, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GPI.

In some embodiments, the level of the RNA is measured by one or more primers or probes specific for RNAs encoded by genes selected from the group consisting of MMP1, DLA-DQB1, IDO1, CCL25, BAX, IL12RB2, PSMB8, STAT2, CSF3R, NOD1, S100A4, TRGC2, IFNA7, TLR4, C7, CD96, TFEB, FCAR, LOC487977, BLNK, GSK3B, and LOC102156778.

In some embodiments, the level of the RNA is measured by a device. In some embodiments, the device is a PCR machine, a hybridization-based array, or a high-throughput sequencing machine. In some embodiments, the device is a NanoString analysis system.

Also disclosed herein is a system comprising
a collection tube;
a preservation solution for a stool sample; and
a set of polynucleotides for measuring levels of target RNAs.

In some embodiments, the system further comprises a device for measuring the levels of target RNAs. In some embodiments, the device is a PCR machine, a hybridization-based array, or a high-throughput sequencing machine. In some embodiments, the system further comprises a NanoString analysis system.

Also disclosed herein are methods for diagnosing, treating, and/or monitoring inflammatory bowel disease (IBD) and other GI disorders, including GI cancers, in humans and companion animals. The invention allows diagnosis, treatment, and/or monitoring of inflammatory bowel disease (IBD) and other GI disorders, including GI cancers, to be made by direct analysis of key immune gene expression using a biological sample (e.g., a fecal sample).

In some aspects, the methods, systems, or platforms of any preceding aspect can be used for diagnosing, treating, and/or monitoring a GI disorder. In some aspects, the methods, systems, or platforms of any preceding aspect can be used for noninvasively monitoring responses to a treatment. In some aspects, the methods, systems, or platforms of any preceding aspect can be used for noninvasively monitoring a GI disorder or monitoring responses to a treatment. The original diagnosis can be made initially through some other approach, such as biopsy. In some embodiments, the GI disorder comprises an enteric infection, GI bacterial overgrowth, GI cancer, inflammatory bowel disease (IBD), or a non-inflammatory GI disease.

In some aspects, disclosed herein is a method for treating inflammatory bowel disease (IBD) in a human or an animal comprising:
obtaining a biological sample from the human or animal;
quantifying an expression level of one or more genes relative to a reference control, wherein the one or more genes are selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567; and
determining that the human or animal is susceptible to or suffering from IBD if the expression level of one or more of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, and CD22 in the biological sample is higher in comparison to the reference control and/or if the expression level of one or more of TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567 in the biological sample is lower in comparison to the reference control; and
and if the human or animal is susceptible to or suffering from IBD: i) administering to the human or animal an effective amount of a therapeutic agent for treating the IBD (e.g., treatments for Crohn's disease or ulcerative colitis), ii) changing the diet of the human or animal, and/or iii) performing a fecal transfaunation in the human or animal.

In some embodiments, the one or more genes are selected from the group consisting of LOC489428, DLA-DQB1, IDO1, CCL25, BAX, IL12RB2, PSMB8, STAT2, CSF3R, NOD1, S100A4, and TRGC2. In some embodiments, the one or more genes are selected from the group consisting of IFNA7, TLR4, C7, CD96, TFEB, FCAR, LOC487977, BLNK, GSK38, and LOC1021567.

In some embodiments, the sample is from a human. In some embodiments, the sample is from a companion animal. In other embodiments, the companion animal is a canine. In some embodiments, the companion animal is a dog. In some embodiments, the companion animal is a feline. In some embodiments, the companion animal is a cat. In other embodiments, the companion animal is a horse.

In some embodiments, the therapeutic agent for treatment of IBD in humans or the animal is selected from a non-absorbable NSAID, an immunosuppressive drug (e.g., methotrexate, budesonide, or prednisone), a biological agent (e.g., anti-TNFα antibody or anti-integrin antibodies), or a Janus kinase inhibitor (e.g., Xeljanz), a probiotic, or an antibiotic.

In some embodiments, the treatment comprises a non-absorbable NSAID (e.g., 5-aminosalicylates).

In some embodiments, the treatment comprises therapeutic monoclonal antibodies (e.g., Humira, Entivyo, or Stelara).

In some embodiments, the treatment comprises Janus kinase inhibitors (e.g., Xeljanz).

In some embodiments, the treatment comprises immunomodulators (e.g., methotrexate, or thiopurines).

In some embodiments, the treatment comprises immune suppressive drugs (e.g., budesonide, or prednisone).

In some embodiments, the treatment comprises antibiotics (metronidazole or ampicillin).

In some embodiments, the biological sample is a fecal sample, a sputum sample, an oral swab sample, a vaginal swab sample, a urethral swab sample, a nasal swab sample, an ocular swab sample, an aural swab sample, or a skin swab sample. In some embodiments, the biological sample is a fecal sample.

In some aspects, disclosed herein is a method for diagnosing or monitoring inflammatory bowel disease (IBD) in a human or an animal comprising:
obtaining a biological sample from the human or animal;
quantifying an expression level of one or more genes relative to a reference control, wherein the one or more genes are selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567; and
determining that the human or animal is susceptible to or suffering from IBD if the expression level of one or more of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, and CD22 in the biological sample is higher in comparison to the reference control and/or if the expression level of one or more of TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567 in the biological sample is lower in comparison to the reference control.

In some embodiments, the method further comprises administering to the human or animal companion animal an effective amount of a therapeutic agent for treating the IBD, changing the diet of the human or animal, or performing a fecal transfaunation, if the human or animal is susceptible to or suffering from IBD.

In some aspects, disclosed herein is a method for monitoring response to a treatment for inflammatory bowel disease (IBD) in a human or an animal comprising:
obtaining a biological sample from the human or animal, wherein the human or animal has received a treatment for IBD;
quantifying an expression level of one or more genes relative to a reference control, wherein the one or more genes are selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567; and
determining that the human or animal is responsive to the treatment if the expression level of one or more of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, and CD22 in the biological sample is lower in comparison to the reference control and/or if the expression level of one or more of TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567 in the biological sample is higher in comparison to the reference control.

Also disclosed herein is a method for treating a gastrointestinal disorder in a human or an animal, comprising:
obtaining a stool sample from the human or animal;
quantifying an expression level of one or more genes relative to a reference control, wherein the one or more genes are selected from the group consisting of MSR1, ARG1, IL11RA, IL2RG, CTSS, RUNX1, CCR1, ADA, TIGIT, MR1, IL27, CRADD, PSMB8, and HLA-DRB3;
determining that the human or animal is susceptible to or suffering from the gastrointestinal disorder if the expression level of one or more genes selected from MSR1 and ARG1 in the biological sample is higher in comparison to the reference control and/or if the expression level of one or more genes selected from IL11RA, IL2RG, CTSS, RUNX1, CCR1, ADA, TIGIT, MR1, IL27, CRADD, PSMB8, and HLA-DRB3 in the biological sample is lower in comparison to the reference control; and
if the human or animal is susceptible to or suffering from the gastrointestinal disorder:
i) administering to the human or animal an effective amount of a therapeutic agent for treating the gastrointestinal disorder, and/or
ii) changing the diet of the human or animal.

Also disclosed herein is a method for diagnosing or monitoring a gastrointestinal disorder in a human or animal, comprising:
obtaining a stool sample from the human or animal;
quantifying an expression level of one or more genes relative to a reference control, wherein the one or more genes are selected from the group consisting of MSR1, ARG1, IL11RA, IL2RG, CTSS, RUNX1, CCR1, ADA, TIGIT, MR1, IL27, CRADD, PSMB8, and HLA-DRB3; and
determining that the human or animal is susceptible to or suffering from the gastrointestinal disorder if the expression level of one or more genes selected from MSR1 and ARG1 in the biological sample is higher in comparison to the reference control and/or if the expression level of one or more genes selected from IL11RA, IL2RG, CTSS, RUNX1, CCR1, ADA, TIGIT, MR1, IL27, CRADD, PSMB8, and HLA-DRB3 in the biological sample is lower in comparison to the reference control.

In some embodiments, the gastrointestinal disorder is IBD, GI cancer, a GI infection, or a non-inflammatory GI disease.

In some aspects, disclosed herein is a method for monitoring response to a treatment for a gastrointestinal disorder in a human or animal, comprising:
obtaining a stool sample from the human or animal, wherein the human or animal has received a treatment for the gastrointestinal disorder;
quantifying an expression level of one or more genes relative to a reference control, wherein the one or more genes are selected from the group consisting of MSR1, ARG1, IL11RA, IL2RG, CTSS, RUNX1, CCR1, ADA, TIGIT, MR1, IL27, CRADD, PSMB8, and HLA-DRB3; and
determining that the human or animal is responsive to the treatment if the expression level of one or more genes selected from MSR1 and ARG1 in the biological sample is higher in comparison to the reference control and/or if the expression level of one or more genes selected from IL11RA, IL2RG, CTSS, RUNX1, CCR1, ADA, TIGIT, MR1, IL27, CRADD, PSMB8, and HLA-DRB3 in the biological sample is lower in comparison to the reference control.

Also disclosed herein is a method for treating a GI cancer (e.g., gastrointestinal lymphoma) in a human or an animal, comprising:
obtaining a stool sample from the human patient or the animal;
quantifying an expression level of one or more genes relative to a reference control, wherein the one or more genes are selected from the group consisting of S100A8, IDO1, CXCL8, FOS, IL1RN, EGR1, CEBPB, EPCAM, S100A9, PLAUR, S100A12, DUSP1, CD74, NFKBIA, LCP1, C1QBP, TNFAIP3, DLA-DRA, DDX5, LGALS3, CCND2, MAP2K2, MEF2C, PSMB9, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GPI;
determining that the human or animal is susceptible to or suffering from the GI cancer (e.g., gastrointestinal lymphoma) if the expression level of one or more genes selected from S100A8, IDO1, CXCL8, FOS, IL1RN, EGR1, CEBPB, S100A9, PLAUR, S100A12, DUSP1, CD74, NFKBIA, LCP1, TNFAIP3, DLA-DRA, DDX5, LGALS3, CCND2, MAP2K2, MEF2C, PSMB9, ANXA1, PTEN, MAPK14, MME, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, and CD28 in the biological sample is higher in comparison to the reference control and/or if the expression level of one or more genes selected from EPCAM, C1QBP, IFNA7, C6, and GPI in the biological sample is lower in comparison to the reference control; and
administering to the human or animal an effective amount of a therapeutic agent for treating the GI cancer (e.g., gastrointestinal lymphoma) if the human or animal is susceptible to or suffering from the GI cancer (e.g., gastrointestinal lymphoma).

Also disclosed herein is a method for diagnosing or monitoring a GI cancer (e.g., gastrointestinal lymphoma) in a human or animal, comprising:
obtaining a stool sample from the human or animal;
quantifying an expression level of one or more genes relative to a reference control, wherein the one or more genes are selected from the group consisting of S100A8, IDO1, CXCL8, FOS, IL1RN, EGR1, CEBPB, EPCAM, S100A9, PLAUR, S100A12, DUSP1, CD74, NFKBIA, LCP1, C1QBP, TNFAIP3, DLA-DRA, DDX5, LGALS3, CCND2, MAP2K2, MEF2C, PSMB9, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GPI; and
determining that the human or animal is susceptible to or suffering from the GI cancer (e.g., gastrointestinal lymphoma) if the expression level of one or more genes selected from S100A8, IDO1, CXCL8, FOS, IL1RN, EGR1, CEBPB, S100A9, PLAUR, S100A12, DUSP1, CD74, NFKBIA, LCP1, TNFAIP3, DLA-DRA, DDX5, LGALS3, CCND2, MAP2K2, MEF2C, PSMB9, ANXA1, PTEN, MAPK14, MME, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, and CD28 in the biological sample is higher in comparison to the reference control and/or if the expression level of one or more genes selected from EPCAM, C1QBP, IFNA7, C6, and GPI in the biological sample is lower in comparison to the reference control.

Also disclosed herein is a method for monitoring response to a treatment for a GI cancer (e.g., gastrointestinal lymphoma) in a human or animal, comprising:
obtaining a stool sample from the human or animal;
quantifying an expression level of one or more genes relative to a reference control, wherein the one or more genes are selected from the group consisting of S100A8, IDO1, CXCL8, FOS, IL1RN, EGR1, CEBPB, EPCAM, S100A9, PLAUR, S100A12, DUSP1, CD74, NFKBIA, LCP1, C1QBP, TNFAIP3, DLA-DRA, DDX5, LGALS3, CCND2, MAP2K2, MEF2C, PSMB9, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GPI; and
determining that the human or animal is responsive to the treatment if the expression level of one or more genes selected from S100A8, IDO1, CXCL8, FOS, IL1RN, EGR1, CEBPB, S100A9, PLAUR, S100A12, DUSP1, CD74, NFKBIA, LCP1, TNFAIP3, DLA-DRA, DDX5, LGALS3, CCND2, MAP2K2, MEF2C, PSMB9, ANXA1, PTEN, MAPK14, MME, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, and CD28 in the biological sample is lower in comparison to the reference control and/or if the expression level of one or more genes selected from EPCAM, C1QBP, IFNA7, C6, and GPI in the biological sample is higher in comparison to the reference control.

### BRIEF DESCRIPTION OF THE DRAWINGS

The accompanying figures, which are incorporated in and constitute a part of this specification, illustrate several aspects described below.

FIG. 1 shows transcriptomic analysis of immune gene expression in fecal samples from dogs with IBD vs healthy dogs (n=8 in each group). mRNA abundance was quantitated in fecal samples, using Nanostring analysis, as described in Methods. The overall abundance of significantly (p<0.05) upregulated and downregulated genes is displayed by volcano plot (left), with downregulated genes (red) and upregulated genes (green) displayed above the horizontal 1.5 fold expression axis. A heat map of most upregulated (green) and downregulated (red) genes is displayed on the right.

FIG. 2 shows transcriptomic analysis of immune gene expression in stool (fecal) samples from dogs with inflammatory bowel disease compared to healthy dogs.

FIG. 3 shows diagnostic gene expression signatures for canine inflammatory bowel disease.

FIG. 4 shows transcriptomic analysis of human stool samples revealing immune gene changes following dietary intervention.

FIG. 5 show differentially expressed genes in human stool samples following dietary intervention.

FIG. 6 shows direct stool transcriptome analysis revealing changes in GI immune transcriptomes in dogs with inflammatory bowel (IBD) disease undergoing dietary intervention.

FIG. 7 shows changes in immune gene expression following implementation of a therapeutic diet in dogs with IBD.

FIG. 8 shows stool transcriptome analysis to identify GI lymphoma in cats.

FIG. 9 shows tubes with stool samples.

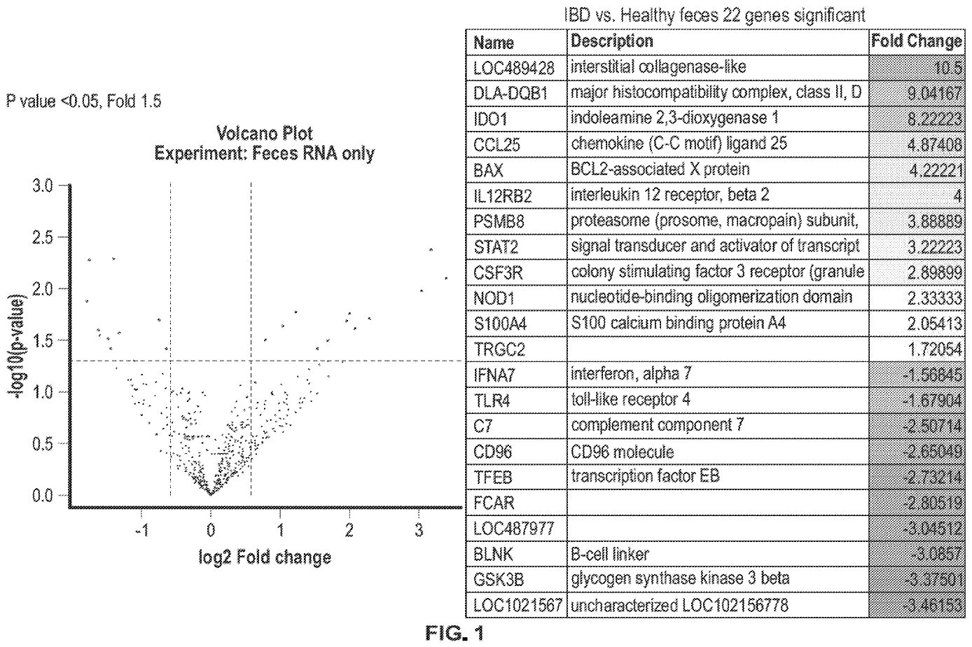

### DETAILED DESCRIPTION

Disclosed herein are methods for diagnosing and treating inflammatory bowel disease (IBD) in humans and companion animals. To date, there are no commercially available assays for accurately identifying IBD in humans or companion animals using single stool samples. Rather, the diagnosis relies on colonoscopy and/or endoscopy with GI biopsy and histopathological interpretation. However, the accuracy of histopathology in diagnosis IBD in humans and animals is not fully reliable, and there is a great deal of variation from one pathologist to another. As disclosed herein, the inventors have developed a novel, non-invasive and much more reliable method for diagnosing and treating IBD in human and companion animals by determining the expression levels of many different genes provided herein in biological samples (e.g., a fecal sample) obtained from humans or companion animals.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “about” as used herein when referring to a measurable value such as an amount, a percentage, and the like, is meant to encompass variations of 20%, ±10%, +5%, or +1% from the measurable value.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. The phrases “concurrent administration”, “administration in combination”, “simultaneous administration” or “administered simultaneously” as used herein, means that the compounds are administered at the same point in time or immediately following one another.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

As used herein, the term “companion animal” refers to those animals traditionally kept for companionship or enjoyment, such as for example, dogs, cats, horses, birds, reptiles, mice, rabbits, hamsters, and the like.

A “composition” is intended to include a combination of active agent and another compound or composition, inert (for example, a detectable agent or label) or active, such as an adjuvant.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “preventing” a disease, a disorder, or unwanted physiological event in a subject refers to the prevention of a disease, a disorder, or unwanted physiological event or prevention of a symptom of a disease, a disorder, or unwanted physiological event.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

As used here, a “substantially pure population” means a population of derived mesenchymal cells that contains at least 99% mesenchymal cells. Cell purification can be accomplished by any means known to one of ordinary skill in the art. For example, a substantially pure population of cells can be achieved by growth of cells or by selection from a less pure population, as described herein.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

“Therapeutically effective amount” or “therapeutically effective dose” of a composition (e.g., a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Systems and Uses Thereof for Extracting and Measuring RNAs in Stool Samples

The gold standard for diagnosis of IBD is GI biopsy and histopathology readout. However, there are numerous studies demonstrating the inability of pathologists to agree on interpretation of GI biopsy samples when looking at same samples. In some cases, it is unable to distinguish between biopsies from healthy individuals and individuals with IBD. This is true of both human IBD and veterinary IBD. Thus, there is a large unmet need for a more objective tool to diagnose IBD through measuring many data points (e.g., RNAs or DNAs) simultaneously, which would eliminate the guesswork and biases of pathologists.

However, evaluation of RNAs directly in stool samples remains a challenge, as in such a harsh environment RNAs would be expected to be rapidly and fully degraded. Thus, a skilled artisan would not suggest a rapid diagnostic platform built around the analysis of stool samples that most people would say RNAs cannot be detected at all.

The instant disclosure has demonstrated systems and methods of isolating and measuring nucleic acids (e.g., RNAs) in stool samples from humans and companion animals. The studies disclosed herein used animal models of IBD and tested human from a diet trial to illustrate that the disclosed systems and method can be used for isolation and measurement of nucleic acids (e.g., RNAs) in stool samples. Uses of the systems, platforms, and/or methods disclosed herein enable the measurement of hundreds or thousands of datapoints in a single small stool sample, providing much greater power and accuracy than measure one or two proteins in stool or in blood.

Accordingly, in some aspects, disclosed herein are methods, systems, platforms, an/or kits relate to using small stool samples and targeted or untargeted transcriptome analysis.

In some aspects, disclosed herein is a method of measuring a level of a nucleic acid (such as mRNA, microRNA, or DNA) in a stool sample, comprising:
a) obtaining a stool sample;
b) extracting the nucleic acid (such as mRNA, microRNA, or DNA) from the stool sample; and
c) measuring the level of the nucleic acid (such as mRNA, microRNA, or DNA).

In some embodiments, the stool sample is obtained from a human or a companion animal.

In some embodiments, the stool sample of step a) is placed or stored in a preservation solution (e.g., an RNA preservation solution).

In some embodiments, step b) further comprises:
adding the stool sample in a tube that comprises a lysis buffer, wherein the lysis buffer comprises β-mercaptoethanol;
mixing the stool sample with the lysis buffer; and
centrifuging the mixed sample thereby obtaining a first supernatant portion.

In some embodiments, the method of any preceding aspect further comprises adding a solution comprising phenol, chloroform, and isoamyl alcohol into the tube prior to adding the stool sample. In some embodiments, the method of any preceding aspect further comprises adding a solution comprising phenol into the tube prior to adding the stool sample.

In some embodiments, the method of any preceding aspect further comprises:
transferring the first supernatant portion into a tube;
mixing the first supernatant portion with an inhibitor removal solution in the tube; and
centrifuging the mixed sample thereby obtaining a second supernatant portion.

In some embodiments, the method of any preceding aspect further comprises
transferring the second supernatant portion into a tube;
mixing the second supernatant portion with a solution comprising binding salts and ethanol; and
passing the mixture through a binding membrane thereby binding the nucleic acid (such as mRNA, microRNA, or DNA) onto the membrane.

In some embodiments, the method of any preceding aspect further comprises adding a DNase to the binding membrane. In some embodiments, the method of any preceding aspect further comprises washing the binding membrane with a washing buffer comprising isopropanol or ethanol. In some embodiments, the method of any preceding aspect further comprises adding water to the binding membrane thereby eluting the RNA in the water.

In some embodiments, the method of any preceding aspect comprises measuring the level of the RNA with an RNA sequencing assay, a hybridization-based array, or a polymerase chain reaction (PCR)-based assay (e.g., reverse transcription PCR (RT-PCR), or real-time PCR). In some embodiments, the RNA is measured by NanoString analysis, real-time PCR, other high-throughput next gen sequencing analysis. Uses of a transcriptome analysis technology (e.g., RNA sequencing, or NanoString targeted panels) allows for the direct analysis in a stool sample of the expression of a panel of GI health and disease related genes. NanoSring analysis can generate panels of 100 to 800 genes, whose expression can be quantitated using the NanoString cassettes and their readers. For full RNA sequencing, several platforms (e.g., Illumina) can be utilized for measuring mRNAs extracted the fecal samples. For targeted analysis of disease detection (e.g., for inflammatory bowel disease), a custom gene panel can be created to focus in on IBD and subtypes of IBD (e.g., Crohn's vs ulcerative colitis) vs healthy subjects. For the global expression concept with NanoString, the cassettes can use all 800 available spots to create a broader net.

In some embodiments, the nucleic acid is an RNA.

In some embodiments, the level of the RNA is measured by one or more (two or more, three or more, four or more, five or more, six or more, seven or more, eight or more, nine or more, or ten or more) primers or probes specific for RNAs encoded by one or more (two or more, three or more, four or more, five or more, six or more, seven or more, eight or more, nine or more, or ten or more) genes selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567.

In some embodiments, the level of the RNA is measured by one or more (two or more, three or more, four or more, five or more, six or more, seven or more, eight or more, nine or more, or ten or more) primers or probes specific for RNAs encoded by one or more (two or more, three or more, four or more, five or more, six or more, seven or more, eight or more, nine or more, or ten or more) genes selected from the group consisting of LOC489428, DLA-DQB1, IDO1, CCL25, BAX, IL12RB2, PSMB8, STAT2, CSF3R, NOD1, S100A4, and TRGC2.

In some embodiments, the level of the RNA is measured by one or more (two or more, three or more, four or more, five or more, six or more, seven or more, eight or more, nine or more, or ten or more) primers or probes specific for RNAs encoded by one or more (two or more, three or more, four or more, five or more, six or more, seven or more, eight or more, nine or more, or ten or more) genes selected from the group consisting of IFNA7, TLR4, C7, CD96, TFEB, FCAR, LOC487977, BLNK, GSK38, and LOC1021567.

In some embodiments, the level of the RNA is measured by one or more (two or more, three or more, four or more, five or more, six or more, seven or more, eight or more, nine or more, or ten or more) primers or probes specific for RNAs encoded by one or more (two or more, three or more, four or more, five or more, six or more, seven or more, eight or more, nine or more, or ten or more) genes selected from the group consisting of TNFRSF13C, CXCL10, SHMT2, CX3CL1, PPARGC1A, DMBT1, FYN, MAVS, CD163, CCL19, SIGIRR, CD84, TXNIP, TXNIP, PRKCD, IL17F, NRAS, C7, C5, CFD, CSF3, LOC483397, and MAPK1. In some embodiments, the accession numbers of the genes are ones in Table 1.

TABLE 1

| | | Accession |
|---|---|---|
| Gene ID | Gene Name | Number |
| TNFRSF13C | TNF receptor superfamily member 13C; TNFRSF13C; ortholog | Q96RJ3 |
| CXCL10 | C-X-C motif chemokine 10; CXCL10; ortholog | P02778 |
| SHMT2 | Serine hydroxymethyltransferase; SHMT2; ortholog | P34897 |
| CX3CL1 | C-X3-C motif chemokine ligand 1; CX3CL1; ortholog | P78423 |
| PPARGC1A | Peroxisome proliferator-activated receptor gamma coactivator 1- | Q9UBK2 |
| | alpha; PPARGC1A; ortholog | |
| DMBT1 | Deleted in malignant brain tumors 1 protein; DMBT1; ortholog | Q9UGM3 |
| FYN | Tyrosine-protein kinase; FYN; ortholog | P06241 |
| MAVS | Mitochondrial antiviral signaling protein; MAVS; ortholog | Q7Z434 |
| CD163 | Scavenger receptor cysteine-rich type 1 protein | Q86VB7 |
| | M130; CD163; ortholog | |
| CCL19 | C-C motif chemokine; CCL19; ortholog | Q99731 |
| SIGIRR | Single Ig and TIR domain containing; SIGIRR; ortholog | Q6IA17 |
| CD84 | CD84 molecule; CD84; ortholog | Q9UIB8 |
| TXNIP | Thioredoxin interacting protein; TXNIP; ortholog | Q9H3M7 |
| TXNIP | Thioredoxin interacting protein; TXNIP; ortholog | Q9H3M7 |
| PRKCD | Protein kinase C delta type; PRKCD; ortholog | Q05655 |
| IL17F | Interleukin 17F; IL17F; ortholog | Q96PD4 |
| NRAS | NRAS proto-oncogene, GTPase; NRAS; ortholog | P01111 |
| C7 | Complement C7; C7; ortholog | P10643 |
| C5 | Complement C5; C5; ortholog | P01031 |
| CFD | Complement Factor D | P00746 |
| CSF3 | Granulocyte colony-stimulating factor; CSF3; ortholog | P09919 |
| LOC483397 | Uncharacterized protein; LOC483397; ortholog | A0A8P0PLR9 |
| MAPK1 | Mitogen-activated protein kinase; MAPK1; ortholog | P28482 |

In some embodiments, the level of the RNA is measured by one or more (two or more, three or more, four or more, five or more, six or more, seven or more, eight or more, nine or more, or ten or more) primers or probes specific for RNAs encoded by one or more (two or more, three or more, four or more, five or more, six or more, seven or more, eight or more, nine or more, or ten or more) genes selected from the group consisting of MSR1, ARG1, IL11RA, IL2RG, CTSS, RUNX1, CCR1, ADA, TIGIT, MR1, L27, CRADD, PSMB, and LA-DRB3. In some embodiments, the accession numbers of the genes are the ones in Table 2.

TABLE 2

| | | Accession |
|---|---|---|
| Gene ID | Gene Name | Number |
| MSR1 | Macrophage scavenger receptor types I and II | P21757 |
| ARG1 | Arginase-1 | P05089 |
| IL11RA | Interleukin-11 receptor subunit alpha | Q14626 |
| IL2RG | Cytokine receptor common subunit gamma | P31785 |
| CTSS | Cathepsin S | P25774 |
| RUNX1 | Runt-related transcription factor 1 | Q01196 |
| CCR1 | C-C chemokine receptor type 1 | P32246 |
| ADA | Adenosine deaminase | P00813 |
| TIGIT | T-cell immunoreceptor with Ig and ITIM domains | Q495A1 |
| MR1 | Probable hydrolase PNKD | Q8N490 |
| IL27 | Interleukin-27 subunit alpha | Q8NEV9 |
| CRADD | Death domain-containing protein CRADD | P78560 |
| PSMB8 | Proteasome subunit beta type-8 | P28062 |
| HLA- | HLA class II histocompatibility antigen, DR beta | P79483 |
| DRB3 | 3 chain | |

In some embodiments, the level of the RNA is measured by one or more (two or more, three or more, four or more, five or more, six or more, seven or more, eight or more, nine or more, or ten or more) primers or probes specific for RNAs encoded by one or more (two or more, three or more, four or more, five or more, six or more, seven or more, eight or more, nine or more, or ten or more) genes selected from the group consisting of S100A8, IDO1, CXCL8, FOS, IL1RN, EGR1, CEBPB, EPCAM, S100A9, PLAUR, S100A12, DUSP5, CD74, NFKBIA, LCP1, C1QBP, TNFAIP3, DLA-DRA, DDX5, LGALS3, CCND2, MAP2K2, MEF2C, PSMB39, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GPI. In some embodiments, the accession numbers of the genes are the ones in Table 3.

TABLE 3

| | | Accession |
|---|---|---|
| Gene ID | Gene Name | Number |
| S100A8 | Protein S100-A8 | P05109 |
| IDO1 | Indoleamine 2,3-dioxygenase 1 | P14902 |
| CXCL8 | Interleukin-8 | P10145 |
| FOS | Proto-oncogene c-Fos | P01100 |
| IL1RN | Interleukin-1 receptor antagonist protein | P18510 |
| EGR1 | Early growth response protein 1 | P18146 |
| CEBPB | CCAAT/enhancer-binding protein beta | P17676 |
| EPCAM | Epithelial cell adhesion molecule | P16422 |
| S100A9 | Protein S100-A9 | P06702 |
| PLAUR | Urokinase plasminogen activator surface receptor | Q03405 |
| S100A12 | Protein S100-A12 | P80511 |
| DUSP1 | Dual specificity protein phophatase 1 | P28562 |
| CD74 | HLA class II histocompatibility antigen gamma chain | P04233 |
| NFKBIA | KF-kappa-B inhibitor alpha | P25963 |
| LCP1 | Plastin-2 | P13796 |
| C1QBP | Complement component 1 Q subcomponent binding protein, | Q07021 |
| | mitochondria | |
| TNFAIP3 | Tumor necrosis factor alpha-induced protein 3 | Q96KP6 |
| DLA- | MHC class II DR alpha chain | A0A8C0RKW1 |
| DRA | | |
| DDX5 | Probable ATP-dependent RNA helicase DD5 | P17844 |
| LGALS3 | Galectin-3 | P17931 |
| CCND2 | G1/S-specific cyclin-D2 | P30279 |
| MAP2K2 | Dual specificity mitogen-activated protein kinase kinase 2 | P36507 |
| MEF2C | Myocyte-specific enhancer factor 2C | Q06413 |
| PSMB9 | Proteasome subunit beta type-9 | P28065 |
| IFNA7 | Interferon alpha-7 | P01567 |
| ANXA1 | Annexin A1 | P04083 |
| PTEN | Phosphatidylinositol 3,4,5-triphosphate 3-phosphatase and dual | P60484 |
| | specific | |
| MAPK14 | Mitogen-activated protein kinase 14 | P49137 |
| MME | Macrophage metalloelastase | P39900 |
| C6 | Complement component C6 | P13671 |
| LYN | Tyrosine-protein kinase Lyn | P07948 |
| STAT3 | Signal transducer and activator of transcription 3 | P40763 |
| CIITA | MHC class II transactivator | P33076 |
| MAPK8 | Mitogen-activated protein kinase 8 | P45983 |
| TRB1 | Tribbles Homolog 1 | Q96RU8 |
| ITGAV | Integrin alpha-V | P06756 |
| ERBB2 | Receptor tyrosine-protein kinase erbB-2 | P04626 |
| NFKB2 | Nuclear factor NK-kappa-B p100 subunit | Q00653 |
| FCER1G | High affinity immunoglobulin epsilon receptor subunit gamma | P30273 |
| HMGCR | 3-hydroxy-3-methylglutaryl-coenzyme A reductase | P04035 |
| RPS6 | 40S ribosomal protein S6 | P62753 |
| HBEGF | Proheparin-binding EGF-like growth factor | Q99075 |
| NDRG1 | Protein NDRG1 | Q92597 |
| STAT2 | Signal transducer and activator of transcription 2 | P52630 |
| APP | Amyloid-beta precursor protein | P05067 |
| VIM | Vimentin | P08670 |
| MAP2K1 | Dual specificity mitogen-activated protein kinase kinase 1 | Q02750 |
| CD28 | T-cell-specific surface glycoprotein CD28 | P10747 |
| GPI | Glucose-6-phosphate isomerase | P06744 |

In some embodiments, the level of the RNA is measured by one or more (two or more, three or more, four or more, five or more, six or more, seven or more, eight or more, nine or more, or ten or more) primers or probes specific for RNAs encoded by one or more (two or more, three or more, four or more, five or more, six or more, seven or more, eight or more, nine or more, or ten or more) genes selected from the group consisting of MMP1, DLA-DQB1, IDO1, CCL25, BAX, IL12RB2, PSMB8, STAT2, CSF3R, NOD1, S100A4, TRGC2, IFNA7, TLR4, C7, CD96, TFEB, FCAR, LOC487977, BLNK, GSK3B, and LOC102156778.

In some embodiments, the level of the RNA is measured by one or more (two or more, three or more, four or more, five or more, six or more, seven or more, eight or more, nine or more, ten or more, twenty or more, thirty or more, forty or more, fifty or more, sixty or more, seventy or more, eighty or more, ninety or more, a hundred or more, three hundreds or more, three hundreds or more, four hundreds or more, five hundreds or more, six hundreds or more, or seven hundreds or more) primers or probes specific for RNAs encoded by one or more (two or more, three or more, four or more, five or more, six or more, seven or more, eight or more, nine or more, ten or more, twenty or more, thirty or more, forty or more, fifty or more, sixty or more, seventy or more, eighty or more, ninety or more, a hundred or more, three hundreds or more, three hundreds or more, four hundreds or more, five hundreds or more, six hundreds or more, or seven hundreds or more) genes selected from Table 4.

Also disclosed herein is a system comprising
a collection tube;
a preservation solution for a stool sample; and
a set of polynucleotides for measuring levels of target nucleic acids (such as mRNA, microRNA, or DNA).

In some embodiments, the primers or probes are specific for RNAs encoded by one or more (two or more, three or more, four or more, five or more, six or more, seven or more, eight or more, nine or more, or ten or more) genes selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567.

In some embodiments, the primers or probes are specific for RNAs encoded by one or more (two or more, three or more, four or more, five or more, six or more, seven or more, eight or more, nine or more, or ten or more) genes selected from the group consisting of LOC489428, DLA-DQB1, IDO1, CCL25, BAX, IL12RB2, PSMB8, STAT2, CSF3R, NOD1, S100A4, and TRGC2.

In some embodiments, the primers or probes are specific for RNAs encoded by one or more (two or more, three or more, four or more, five or more, six or more, seven or more, eight or more, nine or more, or ten or more) genes selected from the group consisting of IFNA7, TLR4, C7, CD96, TFEB, FCAR, LOC487977, BLNK, GSK38, and LOC1021567.

In some embodiments, the primers or probes are specific for RNAs encoded by one or more (two or more, three or more, four or more, five or more, six or more, seven or more, eight or more, nine or more, or ten or more) genes selected from the group consisting of TNFRSF13C, CXCL10, SHMT2, CX3CL1, PPARGC1A, DMBT1, FYN, MAVS, CD163, CCL19, SIGIRR, CD84, TXNIP, TXNIP, PRKCD, IL17F, NRAS, C7, C5, CFD, CSF3, LOC483397, and MAPK1.

In some embodiments, the primers or probes are specific for RNAs encoded by one or more (two or more, three or more, four or more, five or more, six or more, seven or more, eight or more, nine or more, or ten or more) genes selected from the group consisting of MSR1, ARG1, IL11RA, IL2RG, CTSS, RUNX1, CCR1, ADA, TIGIT, MR1, IL27, CRADD, PSMB8, and HLA-DRB3.

In some embodiments, the primers or probes are specific for RNAs encoded by one or more (two or more, three or more, four or more, five or more, six or more, seven or more, eight or more, nine or more, or ten or more) genes selected from the group consisting of S100A8, IDO1, CXCL8, FOS, IL1RN, EGR1, CEBPB, EPCAM, S100A9, PLAUR, S100A12, DUSP1, CD74, NFKBIA, LCP1, C1QBP, TNFAIP3, DLA-DRA, DDX5, LGALS3, CCND2, MAP2K2, MEF2C, PSMB9, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GP.

In some embodiments, the primers or probes are specific for RNAs encoded by one or more (two or more, three or more, four or more, five or more, six or more, seven or more, eight or more, nine or more, or ten or more) genes selected from the group consisting of MMP1, DLA-DQB1, IDO1, CCL25, BAX, IL12RB2, PSMB8, STAT2, CSF3R, NOD1, S100A4, TRGC2, IFNA7, TLR4, C7, CD96, TFEB, FCAR, LOC487977, BLNK, GSK3B, and LOC102156778.

In some embodiments, the level of the RNA is measured by one or more (two or more, three or more, four or more, five or more, six or more, seven or more, eight or more, nine or more, ten or more, twenty or more, thirty or more, forty or more, fifty or more, sixty or more, seventy or more, eighty or more, ninety or more, a hundred or more, three hundreds or more, three hundreds or more, four hundreds or more, five hundreds or more, six hundreds or more, or seven hundreds or more) primers or probes specific for RNAs encoded by one or more (two or more, three or more, four or more, five or more, six or more, seven or more, eight or more, nine or more, ten or more, twenty or more, thirty or more, forty or more, fifty or more, sixty or more, seventy or more, eighty or more, ninety or more, a hundred or more, three hundreds or more, three hundreds or more, four hundreds or more, five hundreds or more, six hundreds or more, or seven hundreds or more) genes selected from Table 4.

In some aspects, disclosed herein uses of the systems or methods of any preceding aspect for treatment, diagnosis, and/or detection of IBD, GI cancers, GI infections, inflammatory diseases, or non-inflammatory GI diseases. In some embodiments, the systems or methods comprise broad screening diagnostic panels and/or more tailored panels specific to a disease. In some embodiments, the methods or systems comprise uses of the primers or probes disclosed herein for measurement of the nucleic acids. In some embodiments, the methods or systems further comprise using NanoString for measurement of the nucleic acids.

### Methods for Treatment and Diagnosis

As noted above, currently the diagnosis of IBD in humans and veterinary species is complicated, time consuming, costly, and often involves the use of endoscopy or colonoscopy procedures, all of which increases cost and risks. Thus, there is a large unmet need for a more objective tool to diagnose IBD through measuring many data points (e.g., RNAs or DNAs) simultaneously, which would eliminate the guesswork and biases of pathologists.

In some aspects, disclosed herein is a method for treating inflammatory bowel disease (IBD) in a human or companion animal comprising:
obtaining a biological sample from the human or companion animal;
quantifying an expression level of one or more genes relative to a reference control, wherein the one or more (two or more, three or more, four or more, five or more, six or more, seven or more, eight or more, nine or more, or ten or more) genes are selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567;
determining that the human or companion animal is susceptible to or suffering from IBD if the expression level of one or more (two or more, three or more, four or more, five or more, six or more, seven or more, eight or more, nine or more, or ten or more) of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, and CD22 in the biological sample is higher in comparison to the reference control and/or if the expression level of one or more of TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567 in the biological sample is lower in comparison to the reference control; and
if the human or companion animal is susceptible to or suffering from IBD:
i) administering to the human or companion animal an effective amount of a therapeutic agent for treating the IBD,
ii) changing the diet of the human or companion animal, or
iii) performing a fecal transfaunation.

The term “reference control” refers to a level in detected in a subject in general or a study population (e.g., healthy control).

In some embodiments, the therapeutic agent for treatment of IBD in humans or the companion animal is selected from an non-absorbable NSAID, an immunosuppressive drug (e.g., methotrexate, budesonide, or prednisone), a biological agent (e.g., anti-TNFα antibody or anti-integrin antibodies), or a Janus kinase inhibitor (e.g., Xeljanz), a probiotic, or an antibiotic.

In some embodiments, the treatment comprises a non-absorbable NSAID (e.g., 5-aminosalicylates).

In some embodiments, the treatment comprises therapeutic monoclonal antibodies (e.g., Humira, Entivyo, or Stelara).

In some embodiments, the treatment comprises Janus kinase inhibitors (e.g., Xeljanz).

In some embodiments, the treatment comprises immunomodulators (e.g., methotrexate, or thiopurines).

In some embodiments, the treatment comprises immune suppressive drugs (e.g., budesonide, or prednisone).

In some embodiments, the quantifying is carried out to detect RNA expression levels. In some embodiments, the quantifying is carried out by one or a combination of Polymerase Chain Reaction, Real Time-Polymerase Chain Reaction, Real Time Reverse Transcriptase-Polymerase Chain Reaction, Real-time quantitative RT-PCR, Northern blot analysis, in situ hybridization, and probe array. In some embodiments, the RNA is measured by NanoString analysis, real-time PCR, other high-throughput next gen sequencing analysis. Uses of a transcriptome analysis technology (e.g., RNA sequencing, or NanoString targeted panels) allows for the direct analysis in a stool sample of the expression of a panel of GI health and disease related genes. NanoString analysis can generate panels of 100 to 800 genes, whose expression can be quantitated using the NanoString cassettes and their readers. For full RNA sequencing, several platforms (e.g., Illumina) can be utilized for measuring mRNAs extracted the fecal samples. For targeted analysis of disease detection (e.g., for inflammatory bowel disease), a custom gene panel can be created to focus in on IBD and subtypes of IBD (e.g., Crohn's vs ulcerative colitis) vs healthy subjects. For the global expression concept with NanoString, the cassettes can use all 800 available spots to create a broader net.

In some embodiments, the quantifying is carried out to detect protein expression levels. In some embodiments, the quantifying is carried out by one or a combination of Western blot, ELISA, flow cytometry, immunohistochemistry, and other methods of detection using antibodies (for example, antibodies to recognize the proteins or polypeptides encoded by the genes provided herein).

In some embodiments, the expression level of the one or more of the genes described herein is about at least 10% higher (for example, at least about 20%, at least about 30%, at least about 40%, at least about 50%, at least about 60%, at least about 70%, at least about 80%, at least about 90%, or at least about a 100% higher or any increase between 10-100% as compared to a reference level, or at least about a 2-fold, or at least about a 3-fold, or at least about a 4-fold, or at least about a 5-fold, or at least about a 10-fold higher, or any increase between 2-fold and 10-fold or greater as compared to a reference level so long as the increase is statistically significant, wherein the genes are selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, and CD22.

In some embodiments, the expression level of the one or more of the genes described herein is about at least 10% lower (for example, at least about 20%, at least about 30%, at least about 40%, at least about 50%, at least about 60%, at least about 70%, at least about 80%, at least about 90%, or at least about a 100% lower or any decrease between 10-100% as compared to a reference level, or at least about a 2-fold, at least about a 3-fold, at least about a 4-fold, at least about a 5-fold, or at least about a 10-fold lower, or any decrease between 2-fold and 10-fold or more as compared to a reference level so long as the decrease is statistically significant, wherein the genes are selected from the group consisting of TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567.

In some aspects, disclosed herein is a method for treating inflammatory bowel disease (IBD) in a human or a companion animal comprising:
obtaining a biological sample from the human or the companion animal;
quantifying an expression level of one or more genes relative to a reference control, wherein the one or more (two or more, three or more, four or more, five or more, six or more, seven or more, eight or more, nine or more, or ten or more) genes are selected from the group consisting of MMP1, DLA-DQB1, IDO1, CCL25, BAX, IL12RB2, PSMB8, STAT2, CSF3R, NOD1, S100A4, TRGC2, IFNA7, TLR4, C7, CD96, TFEB, FCAR, LOC487977, BLNK, GSK3B, and LOC102156778;
determining that the human or companion animal is susceptible to or suffering from IBD if the expression level of one or more (two or more, three or more, four or more, five or more, six or more, seven or more, eight or more, nine or more, or ten or more) of MMP1, DLA-DQB1, IDO1, CCL25, BAX, IL12RB2, PSMB8, STAT2, CSF3R, NOD1, S100A4, and TRGC2 in the biological sample is higher in comparison to the reference control and/or if the expression level of one or more of IFNA7, TLR4, C7, CD96, TFEB, FCAR, LOC487977, BLNK, GSK3B, and LOC102156778 in the biological sample is lower in comparison to the reference control; and
if the human or companion animal is susceptible to or suffering from IBD:
i) administering to the human or companion animal an effective amount of a therapeutic agent for treating the IBD,
ii) changing the diet of the human or companion animal, or
iii) performing a fecal transfaunation.

In some aspects, disclosed herein is a method for treating inflammatory bowel disease (IBD) in a companion animal comprising:
obtaining a biological sample from the companion animal;
quantifying an expression level of one or more genes relative to a reference control, wherein the one or more (two or more, three or more, four or more, five or more, six or more, seven or more, eight or more, nine or more, or ten or more) genes are selected from the group consisting of TNFRSF13C, CXCL10, SHMT2, CX3CL1, PPARGC1A, DMBT1, FYN, MAVS, CD163, CCL19, SIGIRR, CD84, TXNIP, TXNIP, PRKCD, IL17F, NRAS, C7, C5, CFD, CSF3, LOC483397, and MAPK1;
determining that the animal is susceptible to or suffering from IBD if the expression level of one or more (two or more, three or more, four or more, five or more, six or more, seven or more, eight or more, nine or more, or ten or more) of TNFRSF13C, CXCL10, SHMT2, CX3CL1, PPARGC1A, DMBT1, FYN, MAVS, CD163, CCL19, SIGIRR, CD84, TXNIP, TXNIP, PRKCD, IL17F, NRAS, C7, C5, CFD, CSF3, LOC483397, and MAPK1 in the biological sample changes in comparison to the reference control; and
if the animal is susceptible to or suffering from IBD:
i) administering to the companion animal an effective amount of a therapeutic agent for treating the IBD,
ii) changing the diet of the companion animal, or
iii) performing a fecal transfaunation.

In some embodiments, the method of treatment, if the animal is susceptible to or suffering from IBD, comprises administering to the human or companion animal an effective amount of a therapeutic agent for treating IBD. In some embodiments, the method of treatment, if the animal is susceptible to or suffering from IBD, comprises changing the diet of the human or companion animal. In some embodiments, the method of treatment, if the animal is susceptible to or suffering from IBD, comprises performing a fecal transfaunation.

In some embodiments, the one or more (two or more, three or more, four or more, five or more, six or more, seven or more, eight or more, nine or more, or ten or more) genes are selected from the group consisting of LOC489428, DLA-DQB1, IDO1, CCL25, BAX, IL12RB2, PSMB8, STAT2, CSF3R, NOD1, S100A4, and TRGC2. In some embodiments, the one or more genes (two or more, three or more, four or more, five or more, or six or more) are selected from the group consisting of LOC489428, DLA-DQB1, IDO1, CCL25, BAX, and IL12RB2.

In some embodiments, the one or more genes (two or more, three or more, four or more, five or more, or six or more) are selected from the group consisting of IFNA7, TLR4, C7, CD96, TFEB, FCAR, LOC487977, BLNK, GSK38, and LOC1021567. In some embodiments, the one or more genes (two or more, three or more, or four or more) are selected from the group consisting of TFEB, FCAR, LOC487977, GSK38, and LOC1021567.

In some embodiments, the companion animal is a canine. In some embodiments, the companion animal is a dog. In some embodiments, the companion animal is a feline. In some embodiments, the companion animal is a cat. In some embodiments, the companion animal is a horse.

In some embodiments, instead of a companion animal, the subject may be a human.

In some embodiments, the biological sample is a fecal sample, a sputum sample, an oral swab sample, a vaginal swab sample, a urethral swab sample, a nasal swab sample, an ocular swab sample, an aural swab sample, or a skin swab sample. In some embodiments, the biological sample is a fecal sample.

In some embodiments, the therapeutic agent is selected from an antibiotic, an immunosuppressive agent, or a probiotic. In some embodiments, the therapeutic agent is an antibiotic. In some embodiments, the therapeutic agent is an immunosuppressive agent. In some embodiments, the therapeutic agent is a probiotic.

In some embodiments, the antibiotic comprises metronidazole, tylosin, or ampicillin. In some embodiments, the antibiotic is metronidazole. In some embodiments, the antibiotic is tylosin. In some embodiments, the antibiotic is ampicillin.

In some embodiments, the immunosuppressive agent comprises prednisone, prednisolone, budesonide, cyclosporine, mycophenolate, or chlorambucil. In some embodiments, the immunosuppressive agent is prednisone. In some embodiments, the immunosuppressive agent is cyclosporine. In some embodiments, the immunosuppressive agent is mycophenolate. In some embodiments, the immunosuppressive agent is chlorambucil.

In some embodiments, the method further comprises administering to the human or companion animal an effective amount of a therapeutic agent for treating the IBD or changing the diet of the human or companion animal, if the animal is susceptible to or suffering from IBD.

In some embodiments, the fecal sample is a fresh sample. In some embodiments, the fecal sample can be a frozen sample.

In some embodiments, the method further comprises a step for processing the fecal sample to produce a fecal bacterial suspension. In some embodiments, the method further comprises a step wherein the fecal bacterial suspension is centrifuged to obtain a bacterial pellet. In some embodiments, the bacterial pellet is resuspended and incubated with a detecting antibody.

In some aspects, disclosed herein is a method for diagnosing inflammatory bowel disease (IBD) in a human or companion animal comprising:
obtaining a biological sample from the human or companion animal;
quantifying an expression level of one or more (two or more, three or more, four or more, five or more, six or more, seven or more, eight or more, nine or more, or ten or more) genes relative to a reference control, wherein the one or more genes are selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567; and
determining that the animal is susceptible to or suffering from IBD if the expression level of one or more (two or more, three or more, four or more, five or more, six or more, seven or more, eight or more, nine or more, or ten or more) of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, and CD22 in the biological sample is higher in comparison to the reference control and/or if the expression level of one or more (two or more, three or more, four or more, five or more, six or more, seven or more, eight or more, nine or more, or ten or more) of TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567 in the biological sample is lower in comparison to the reference control.

In some aspects, disclosed herein is a method for diagnosing inflammatory bowel disease (IBD) in a human or companion animal comprising:
obtaining a biological sample from the human or companion animal;
quantifying an expression level of one or more genes relative to a reference control, wherein the one or more (two or more, three or more, four or more, five or more, six or more, seven or more, eight or more, nine or more, or ten or more) genes are selected from the group consisting of MMP1, DLA-DQB1, IDO1, CCL25, BAX, IL12RB2, PSMB8, STAT2, CSF3R, NOD1, S100A4, TRGC2, IFNA7, TLR4, C7, CD96, TFEB, FCAR, LOC487977, BLNK, GSK3B, and LOC102156778; and
determining that the animal is susceptible to or suffering from IBD if the expression level of one or more (two or more, three or more, four or more, five or more, six or more, seven or more, eight or more, nine or more, or ten or more) of MMP1, DLA-DQB1, IDO1, CCL25, BAX, IL12RB2, PSMB8, STAT2, CSF3R, NOD1, S100A4, and TRGC2 in the biological sample is higher in comparison to the reference control and/or if the expression level of one or more of IFNA7, TLR4, C7, CD96, TFEB, FCAR, LOC487977, BLNK, GSK3B, and LOC102156778 in the biological sample is lower in comparison to the reference control.

In some aspects, disclosed herein is a method for diagnosing inflammatory bowel disease (IBD) in a human or companion animal comprising:
obtaining a biological sample from the human or companion animal;
quantifying an expression level of one or more genes relative to a reference control, wherein the one or more (two or more, three or more, four or more, five or more, six or more, seven or more, eight or more, nine or more, or ten or more) genes are selected from the group consisting of TNFRSF13C, CXCL10, SHMT2, CX3CL1, PPARGC1A, DMBT1, FYN, MAVS, CD163, CCL19, SIGIRR, CD84, TXNIP, TXNIP, PRKCD, IL17F, NRAS, C7, C5, CFD, CSF3, LOC483397, and MAPK1;
determining that the animal is susceptible to or suffering from IBD if the expression level of one or more (two or more, three or more, four or more, five or more, six or more, seven or more, eight or more, nine or more, or ten or more) of TNFRSF13C, CXCL10, SHMT2, CX3CL1, PPARGC1A, DMBT1, FYN, MAVS, CD163, CCL19, SIGIRR, CD84, TXNIP, TXNIP, PRKCD, IL17F, NRAS, C7, C5, CFD, CSF3, LOC483397, and MAPK1 in the biological sample changes in comparison to the reference control.

In some embodiments, the method further comprises administering to the human or companion animal an effective amount of a therapeutic agent for treating the IBD, changing the diet of the human or companion animal, or performing a fecal transfaunation, if the animal is susceptible to or suffering from IBD.

Also disclosed herein is a method for treating a gastrointestinal disorder in a human or a companion animal, comprising:
obtaining a stool sample from the human or companion animal;
quantifying an expression level of one or more (two or more, three or more, four or more, five or more, six or more, seven or more, eight or more, nine or more, or ten or more) genes relative to a reference control, wherein the one or more (two or more, three or more, four or more, five or more, six or more, seven or more, eight or more, nine or more, or ten or more) genes are selected from the group consisting of MSR1, ARG1, IL11RA, IL2RG, CTSS, RUNX1, CCR1, ADA, TIGIT, MR1, IL27, CRADD, PSMB8, and HLA-DRB3;
determining that the human or companion animal is susceptible to or suffering from the gastrointestinal disorder if the expression level of one or more genes selected from MSR1 and ARG1 in the biological sample is higher in comparison to the reference control and/or if the expression level of one or more (two or more, three or more, four or more, five or more, six or more, seven or more, eight or more, nine or more, or ten or more) genes selected from IL11RA, IL2RG, CTSS, RUNX1, CCR1, ADA, TIGIT, MR1, IL27, CRADD, PSMB8, and HLA-DRB3 in the biological sample is lower in comparison to the reference control; and
if the human is susceptible to or suffering from the gastrointestinal disorder:
i) administering to the human or companion animal an effective amount of a therapeutic agent for treating the gastrointestinal disorder, and/or
ii) changing the diet of the human or companion animal.

In some embodiments, the human or companion animal is susceptible to or suffering from IBD, GI cancers, GI infections, or non-inflammatory GI diseases. In some embodiments, the human is susceptible to or suffering from Crohn's disease, ulcerative colitis, or microscopic colitis. In some embodiments, the human susceptible to or suffering from Crohn's disease is treated with an anti-inflammatory medicine (e.g., sulfasalazine, mesalamine, balsalazide, corticosteroids, or a nonsteroidal anti-inflammatory drug), an antibiotic, an antidiarrheal medication, or probiotics.

Also disclosed herein is a method for diagnosing a gastrointestinal disorder in a human or companion animal, comprising:
obtaining a stool sample from the human or companion animal;
quantifying an expression level of one or more (two or more, three or more, four or more, five or more, six or more, seven or more, eight or more, nine or more, or ten or more) genes relative to a reference control, wherein the one or more (two or more, three or more, four or more, five or more, six or more, seven or more, eight or more, nine or more, or ten or more) genes are selected from the group consisting of MSR1, ARG1, IL11RA, IL2RG, CTSS, RUNX1, CCR1, ADA, TIGIT, MR1, IL27, CRADD, PSMB8, and HLA-DRB3; and
determining that the human or companion animal is susceptible to or suffering from the gastrointestinal disorder if the expression level of one or more genes selected from MSR1 and ARG1 in the biological sample is higher in comparison to the reference control and/or if the expression level of one or more (two or more, three or more, four or more, five or more, six or more, seven or more, eight or more, nine or more, or ten or more) genes selected from IL11RA, IL2RG, CTSS, RUNX1, CCR1, ADA, TIGIT, MR1, IL27, CRADD, PSMB8, and HLA-DRB3 in the biological sample is lower in comparison to the reference control.

Also disclosed herein is a method for treating gastrointestinal lymphoma in a human or companion animal, comprising:
obtaining a stool sample from the human or companion animal;
quantifying an expression level of one or more genes relative to a reference control, wherein the one or more (two or more, three or more, four or more, five or more, six or more, seven or more, eight or more, nine or more, or ten or more) genes are selected from the group consisting of S100A8, IDO1, CXCL8, FOS, IL1RN, EGR1, CEBPB, EPCAM, S100A9, PLAUR, S100A12, DUSP1, CD74, NFKBIA, LCP1, C1QBP, TNFAIP3, DLA-DRA, DDX5, LGALS3, CCND2, MAP2K2, MEF2C, PSMB9, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GPI;
determining that the human or companion animal is susceptible to or suffering from gastrointestinal lymphoma if the expression level of one or more (two or more, three or more, four or more, five or more, six or more, seven or more, eight or more, nine or more, or ten or more) genes selected from S100A8, IDO1, CXCL8, FOS, IL1RN, EGR1, CEBPB, S100A9, PLAUR, S100A12, DUSP1, CD74, NFKBIA, LCP1, TNFAIP3, DLA-DRA, DDX5, LGALS3, CCND2, MAP2K2, MEF2C, PSMB9, ANXA1, PTEN, MAPK14, MME, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, and CD28 in the biological sample is higher in comparison to the reference control and/or if the expression level of one or more (two or more, three or more, four or more, or five or more) genes selected from EPCAM, C1QBP, IFNA7, C6, and GPI in the biological sample is lower in comparison to the reference control; and
administering to the human or companion animal an effective amount of a therapeutic agent for treating the gastrointestinal lymphoma if the human or companion animal is susceptible to or suffering from gastrointestinal lymphoma.

Also disclosed herein is a method for diagnosing gastrointestinal lymphoma in a human or companion animal, comprising:
obtaining a stool sample from the human or companion animal;
quantifying an expression level of one or more (two or more, three or more, four or more, five or more, six or more, seven or more, eight or more, nine or more, or ten or more) genes relative to a reference control, wherein the one or more (two or more, three or more, four or more, five or more, six or more, seven or more, eight or more, nine or more, or ten or more) genes are selected from the group consisting of S100A8, IDO1, CXCL8, FOS, IL1RN, EGR1, CEBPB, EPCAM, S100A9, PLAUR, S100A12, DUSP1, CD74, NFKBIA, LCP1, C1QBP, TNFAIP3, DLA-DRA, DDX5, LGALS3, CCND2, MAP2K2, MEF2C, PSMB9, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GPI; and
determining that the human or companion animal is susceptible to or suffering from gastrointestinal lymphoma if the expression level of one or more (two or more, three or more, four or more, five or more, six or more, seven or more, eight or more, nine or more, or ten or more) genes selected from S100A8, IDO1, CXCL8, FOS, IL1RN, EGR1, CEBPB, S100A9, PLAUR, S100A12, DUSP1, CD74, NFKBIA, LCP1, TNFAIP3, DLA-DRA, DDX5, LGALS3, CCND2, MAP2K2, MEF2C, PSMB9, ANXA1, PTEN, MAPK14, MME, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, and CD28 in the biological sample is higher in comparison to the reference control and/or if the expression level of one or more (two or more, three or more, four or more, or five or more) genes selected from EPCAM, C1QBP, IFNA7, C6, and GPI in the biological sample is lower in comparison to the reference control.

In some aspects, disclosed herein are uses of the system or platform of any preceding aspect for treatment and/or diagnosis of IBD comprising
obtaining a stool sample from the human or companion animal;
quantifying an expression level of one or more (two or more, three or more, four or more, five or more, six or more, seven or more, eight or more, nine or more, ten or more, twenty or more, thirty or more, forty or more, fifty or more, sixty or more, seventy or more, eighty or more, ninety or more, a hundred or more, three hundreds or more, three hundreds or more, four hundreds or more, five hundreds or more, six hundreds or more, or seven hundreds or more) genes selected from Table 4; and
determining that the human or companion animal is susceptible to or suffering from IBD if the expression level of one or more (two or more, three or more, four or more, five or more, six or more, seven or more, eight or more, nine or more, ten or more, twenty or more, thirty or more, forty or more, fifty or more, sixty or more, seventy or more, eighty or more, ninety or more, a hundred or more, three hundreds or more, three hundreds or more, four hundreds or more, five hundreds or more, six hundreds or more, or seven hundreds or more) genes selected from Table 4 in the biological sample is lower in comparison to the reference control.

In some embodiments, the accession numbers of the genes provided herein are those listed in Table 4.

TABLE 4

| | | log2FoldChange |
|---|---|---|
| Symbol | accession number | (IBD vs Healthy) |
| ABCB1 | NM_001003215.1 | 0.489584 |
| ABL1 | XM_005625232.1 | 0 |
| ACOD1 | XM_542615.6 | −0.36257 |
| ACVR1 | XM_022414922.1 | 0.045088 |
| ADA | XM_005635097.2 | 0.799701 |
| ADAMTS2 | XM_843844.4 | −0.05648 |
| ADGRE5 | NM_001048110.1 | 0.370478 |
| ADORA2A | NM_001003278.1 | 0.598956 |
| ADORA3 | NM_001003178.1 | −0.06936 |
| AICDA | NM_001003380.1 | 0.164445 |
| AIRE | XM_022413324.1 | 0.553598 |
| AKT1 | XM_548000.4 | −0.16128 |
| AKT3 | XM_022421141.1 | 0.807355 |
| ALCAM | NM_001313804.1 | −0.24553 |
| AMBP | XM_538807.6 | −0.05881 |
| ANKRD22 | XM_005636647.3 | 0.5517 |
| ANXA1 | XM_533524.2 | 0.241501 |
| APC | XM_014111995.2 | 0.104843 |
| APOE | XM_022425020.1 | 0.1668 |
| APP | NM_001293279.1 | 0.152003 |
| AR | NM_001003053.1 | 0 |
| ARG1 | XM_532053.2 | 0 |
| ARG2 | XM_022421753.1 | −0.40696 |
| ATF1 | XR_295821.1 | −0.09077 |
| ATF2 | XM_005640334.3 | 0 |
| ATG10 | XM_005618157.3 | 0.10806 |
| ATG12 | XM_022425133.1 | −0.1835 |
| ATG16L1 | XM_845571.5 | 0.304855 |
| ATG5 | XM_014118180.2 | 0 |
| ATG7 | XM_022406360.1 | 0.859822 |
| ATM | NM_001130828.1 | 0 |
| ATRX | XM_014111550.2 | −0.05663 |
| AXL | XM_022405907.1 | 0 |
| B2M | NM_001284479.1 | 0.437495 |
| BATF | XM_849670.1 | 1 |
| BAX | NM_001003011.1 | 2.078003 |
| BCL10 | XM_547304.2 | 0.654503 |
| BCL2 | NM_001002949.1 | −0.04389 |
| BCL2L1 | NM_001003072.1 | −0.10673 |
| BCL6 | NM_001195404.1 | 0.011229 |
| BCR | XM_014108205.2 | 0.360423 |
| BHLHE40 | XM_541795.6 | 0.444785 |
| BID | NM_001251938.1 | −0.00893 |
| BIRC5 | NM_001003348.1 | 0.347923 |
| BLK | XM_543206.4 | −0.26298 |
| BLNK | XM_005637573.3 | −1.6256 |
| BMI1 | NM_001287063.1 | 0 |
| BRAF | XM_022403736.1 | −0.15446 |
| BRCA1 | NM_001013416.1 | −0.1835 |
| BRCA2 | NM_001006653.4 | 0 |
| BST1 | XM_545938.6 | 0.136663 |
| BST2 | XM_860510.5 | −0.11134 |
| BTK | XM_005641566.2 | 0.192019 |
| BTLA | XM_545097.2 | 0.078003 |
| C1QA | XM_535367.6 | 0.200366 |
| C1QB | XM_544507.5 | −0.12553 |
| C1QBP | XM_546568.6 | −0.15147 |
| C1R | XM_534901.6 | −0.14917 |
| C1S | XM_005637210.3 | 0.035189 |
| C2 | XM_014118342.2 | 0.168295 |
| C3 | XM_022407130.1 | 0.402965 |
| C3AR1 | XM_005637196.3 | −0.53945 |
| C4BPA | XM_003434950.4 | 0.361795 |
| C5 | XM_022425033.1 | −0.05242 |
| C6 | XM_022417885.1 | 0.212885 |
| C7 | XM_022417892.1 | −1.32604 |
| C8A | XM_003639022.3 | −0.18569 |
| C8B | XM_536694.6 | −0.05242 |
| C8G | XM_014116920.2 | −0.31603 |
| C9 | XM_005619370.3 | 0 |
| CALHM6 | XM_850260.5 | 0 |
| CAMP | NM_001003359.1 | 0.384664 |
| CARD11 | XM_005621150.3 | 0 |
| CARD9 | XM_844178.1 | −0.17243 |
| CASP10 | XM_005640530.3 | 1.254101 |
| CASP3 | NM_001003042.1 | 0.520916 |
| CASP8 | NM_001048029.1 | 1.015942 |
| CCL1 | NM_001005252.1 | −0.06301 |
| CCL13 | NM_001003966.1 | 0.63743 |
| CCL14 | XM_537723.6 | 0.351126 |
| CCL16 | XM_537724.6 | 0.985353 |
| CCL17 | NM_001003051.1 | −0.54507 |
| CCL19 | NM_001005256.1 | 0 |
| CCL2 | NM_001003297.1 | −0.29155 |
| CCL20 | NM_001005254.1 | −0.05242 |
| CCL21 | NM_001005258.1 | 0 |
| CCL22 | XM_003433778.2 | 0.117173 |
| CCL23 | XM_537722.6 | 0 |
| CCL24 | NM_001003967.1 | 1.289507 |
| CCL25 | NM_001005259.1 | 2.285128 |
| CCL26 | NM_001005253.1 | 0 |
| CCL27 | NM_001003968.1 | 0.2358 |
| CCL28 | NM_001005257.1 | −0.05716 |
| CCL3 | NM_001005251.1 | 0 |
| CCL4 | NM_001005250.1 | −0.04064 |
| CCL5 | NM_001003010.2 | −0.28526 |
| CCL7 | NM_001010960.1 | 0.071577 |
| CCL8 | NM_001005255.1 | 0.117173 |
| CCND1 | NM_001005757.1 | −0.05242 |
| CCND2 | XM_849493.1 | 0 |
| CCND3 | XM_859764.3 | −0.04786 |
| CCR1 | NM_001038606.1 | 0 |
| CCR10 | XM_844228.3 | 0.157961 |
| CCR2 | XM_541906.1 | −1.36257 |
| CCR3 | NM_001005261.1 | 0 |
| CCR4 | NM_001003020.1 | 0.127178 |
| CCR5 | NM_001012342.2 | 0 |
| CCR6 | XM_846017.4 | −0.03634 |
| CCR7 | XM_548131.2 | 0 |
| CCR9 | NM_001284476.1 | −0.29844 |
| CCRL2 | XM_541904.5 | 0.086737 |
| CD14 | XM_843653.5 | 0.117418 |
| CD160 | XM_005630717.1 | 0 |
| CD163 | NM_001048020.1 | 0 |
| CD164 | XM_532256.6 | 0.098334 |
| CD180 | XM_544362.6 | 0.444785 |
| CD19 | XM_005621381.3 | 0 |
| CD1A6 | NM_001128837.1 | 0 |
| CD1B | NM_001130830.1 | 0.064752 |
| CD1C | NM_001128836.1 | 0 |
| CD1D | XM_005640956.2 | 0 |
| CD1E | XM_005640914.2 | −0.71348 |
| CD2 | XM_844126.1 | 0.830075 |
| CD200 | XM_005639479.2 | 0 |
| CD207 | XM_850409.2 | 0 |
| CD209 | NM_001130832.1 | −0.00893 |
| CD22 | XM_862798.3 | 0.424748 |
| CD244 | XM_005640944.1 | −0.26298 |
| CD247 | XM_005622970.1 | 0 |
| CD27 | XM_849371.1 | 0.146991 |
| CD274 | XM_541302.2 | −0.05242 |
| CD276 | XM_849111.1 | −1.1281 |
| CD28 | NM_001003087.1 | 0 |
| CD34 | NM_001003341.1 | 0 |
| CD36 | NM_001177734.2 | 0.136663 |
| CD37 | XM_541497.4 | 0 |
| CD38 | NM_001003143.1 | 0.053265 |
| CD3D | XM_536556.2 | 0 |
| CD3E | NM_001003379.1 | 0.215954 |
| CD3EAP | XM_022405335.1 | 0.374396 |
| CD3G | XM_005619711.3 | 0.279842 |
| CD4 | NM_001003252.1 | −0.16424 |
| CD40 | NM_001002982.1 | 0 |
| CD40LG | NM_001002981.1 | 0 |
| CD44 | NM_001197022.1 | −0.26298 |
| CD47 | NM_001080721.1 | 0.147487 |
| CD48 | XM_545759.5 | 0 |
| CD5 | XM_005631690.3 | −0.0857 |
| CD53 | XM_003639132.2 | 0.117173 |
| CD55 | XM_022420853.1 | 0.048409 |
| CD58 | XM_014120568.2 | 0.116899 |
| CD59 | XM_022405336.1 | 0.13493 |
| CD6 | XM_014121151.1 | 0 |
| CD63 | XM_005625462.1 | −0.01207 |
| CD68 | XM_022418524.1 | 0.033165 |
| CD7 | XM_844435.4 | −0.15612 |
| CD70 | XM_542136.4 | −0.00893 |
| CD74 | XM_005619298.3 | 1.282765 |
| CD79A | NM_001313834.1 | 0.124689 |
| CD79B | XM_005624254.3 | 0 |
| CD80 | NM_001003147.1 | 0 |
| CD81 | XM_014120935.1 | 0.104843 |
| CD83 | XM_847554.5 | 0.672195 |
| CD84 | XM_005640884.3 | 0 |
| CD86 | NM_001003146.1 | 0 |
| CD8A | XM_850452.1 | −0.06622 |
| CD8B | XM_859954.4 | −0.29992 |
| CD9 | XM_005637440.2 | 0 |
| CD96 | XM_545094.2 | −1.40626 |
| CD99 | XM_014111551.1 | 0.454566 |
| CDH1 | NM_001287125.1 | 0.523562 |
| CDH5 | XM_546894.6 | 0 |
| CDK1 | XM_014112825.1 | 0.315502 |
| CDK4 | XM_022424104.1 | 0 |
| CDKN1A | XM_532125.2 | 0.644094 |
| CDKN2A | XM_003431598.2 | −0.42866 |
| CDKN2B | NM_001146269.1 | 0.096862 |
| CDKN2C | XM_005629013.2 | −0.05242 |
| CEACAM1 | NM_001097557.1 | 0.742512 |
| CEBPA | XM_005616767.1 | −0.05098 |
| CEBPB | XM_005635170.3 | 1.421701 |
| CFB | XM_003431676.3 | 0.152003 |
| CFD | XM_542213.6 | 0.006165 |
| CFI | XM_014109975.2 | −0.2475 |
| CFP | XM_548978.6 | −0.21818 |
| CH25H | NM_001313827.1 | −0.21503 |
| CHEK2 | XM_022410496.1 | −0.34346 |
| CHUK | XM_005637611.3 | 0.546603 |
| CIITA | XM_547128.2 | −0.035 |
| CISH | XM_541873.6 | 0.035979 |
| CKLF | NM_001252340.1 | −0.45003 |
| CLDN1 | XM_845155.5 | 0.119446 |
| CLEC4A | XM_014108320.2 | 0.633967 |
| CLEC5A | XM_014119959.2 | −0.36257 |
| CLEC7A | XM_022411028.1 | 0.485427 |
| CLU | NM_001003370.1 | 0.300395 |
| CMA1 | NM_001013424.1 | −0.33997 |
| CMKLR1 | XM_022410212.1 | 0.559427 |
| CNP | XM_005624476.3 | 0.113178 |
| COL1A1 | NM_001003090.1 | 0.460552 |
| COL3A1 | XM_845916.1 | 0 |
| COLEC12 | XM_022421537.1 | −0.12278 |
| CPA3 | XM_542828.6 | 0 |
| CR1L | XM_005622317.2 | 0.0199 |
| CR2 | XM_005622319.1 | −0.52696 |
| CREB1 | XM_014110992.2 | 0.343699 |
| CREB5 | XM_022427523.1 | −0.23397 |
| CREBBP | XM_003434864.4 | 0 |
| CRP | XM_545746.2 | 0 |
| CSF1 | XM_005621807.2 | 0 |
| CSF1R | XM_546306.6 | −0.63941 |
| CSF2 | NM_001003245.1 | 0 |
| CSF2RB | XM_538397.6 | −0.08955 |
| CSF3 | XM_022423955.1 | 0.31797 |
| CSF3R | XM_005628934.3 | 1.53555 |
| CTLA4 | NM_001003106.1 | −0.31818 |
| CTSG | XM_014115810.2 | −0.31223 |
| CTSH | XM_536212.3 | −0.05772 |
| CTSS | NM_001002938.1 | 0.736966 |
| CTSW | XM_540846.5 | 0 |
| CX3CL1 | NM_001284456.1 | 0 |
| CX3CR1 | NM_001284491.1 | 0.011229 |
| CXCL10 | NM_001010949.1 | 0.917538 |
| CXCL11 | XM_003640114.2 | 0.011825 |
| CXCL12 | NM_001128097.1 | −0.63176 |
| CXCL13 | XM_845089.3 | −0.38565 |
| CXCL14 | XM_005626560.1 | −0.05242 |
| CXCL16 | XM_844211.3 | 0.155278 |
| CXCL8 | NM_001003200.1 | 0.747854 |
| CXCR1 | XM_005640640.2 | 0.495411 |
| CXCR2 | NM_001003151.2 | −0.47335 |
| CXCR3 | NM_001011887.1 | 0 |
| CXCR4 | NM_001048026.1 | 0 |
| CXCR5 | XM_546496.3 | 0.090289 |
| CXCR6 | XM_846798.4 | −0.89308 |
| CYBB | NM_001100291.1 | 1.088091 |
| CYFIP2 | XM_005619254.3 | 0.024453 |
| CYLD | XM_005617569.3 | 0 |
| DDR1 | XM_022425821.1 | 0.260892 |
| DDX5 | NM_001286941.1 | 0.651785 |
| DDX58 | XM_003639385.2 | 0 |
| DEFB1 | NM_001113713.1 | −0.14101 |
| DLA-12 | NM_001014379.1 | 0.590124 |
| DLA-64 | NM_001014378.1 | 0.02057 |
| DLA-79 | NM_001020810.1 | 0.084889 |
| DLA88 | NM_001014767.1 | 0.590314 |
| DLA-DMA | NM_001048099.1 | −0.12587 |
| DLA-DMB | NM_001271070.1 | 0.301867 |
| DLA-DOB | NM_001048127.1 | 0 |
| DLA-DQA1 | NM_001011726.1 | 0.63743 |
| DLA-DQB1 | NM_001014381.1 | 3.176589 |
| DLA-DRA | NM_001011723.1 | 0.787735 |
| DMBT1 | XM_014109111.2 | 0 |
| DNMT3A | XM_022404353.1 | −0.36257 |
| DOCK2 | XM_546246.5 | 0.252512 |
| DOK1 | XM_540216.6 | 0.350534 |
| DOK2 | XM_543259.6 | 0.024453 |
| DUSP1 | XM_014112785.1 | −0.05445 |
| DUSP6 | XM_860257.1 | 0.029294 |
| EBI3 | XM_005633060.3 | −0.26298 |
| EGFR | XM_533073.2 | 0 |
| EGR1 | XM_846145.3 | 0.230006 |
| ELANE | NM_001003378.2 | 0 |
| ENTPD1 | XM_005637555.3 | −0.79553 |
| EOMES | XM_845645.3 | 0 |
| EP300 | XM_022424377.1 | 0 |
| EPCAM | XM_005626238.1 | 0.513102 |
| EPHA5 | XM_005628243.2 | −0.26838 |
| EPSTI1 | XM_005633925.1 | 1.029747 |
| ERBB2 | NM_001003217.1 | 0.892687 |
| ERBB3 | XM_538226.6 | −0.15612 |
| ERBB4 | XM_003434277.4 | 0 |
| ESR1 | NM_001286958.1 | −0.36257 |
| ESR2 | XM_861041.4 | 0 |
| ETS1 | XM_546405.5 | −0.03717 |
| F13A1 | XM_022414421.1 | −0.31835 |
| FABP7 | XM_533484.6 | 0.015915 |
| FADD | XM_005631743.1 | −0.04064 |
| FAS | XM_005636650.1 | 0.269698 |
| FASLG | XM_848916.1 | 1 |
| FAT1 | XM_022404178.1 | 0.38807 |
| FBXW11 | XM_861445.4 | 0 |
| FCAR | XM_014116440.1 | −1.4881 |
| FCER1A | NM_001110766.2 | 0.416789 |
| FCER1G | NM_001003171.2 | 0.241862 |
| FCER2 | XM_022407108.1 | 0.253573 |
| FCGR1A | XM_014120558.2 | −0.13486 |
| FCRL2 | XM_849766.5 | −0.4736 |
| FGFR1 | XM_014120029.1 | −0.00502 |
| FGFR3 | XM_005618534.2 | 0 |
| FLT3 | NM_001020811.1 | 0 |
| FLT3LG | NM_001003350.1 | 0 |
| FN1 | XM_536059.2 | 0.576029 |
| FOS | XM_547914.5 | 0.222392 |
| FOXA1 | XM_847261.4 | 0 |
| FOXP3 | NM_001168461.1 | 0.453005 |
| FPR2 | XM_005616212.2 | −0.11259 |
| FYN | XM_005627719.2 | 0 |
| GATA3 | XM_844060.1 | −1.14018 |
| GBP5 | XM_547291.4 | 0 |
| GFAP | XM_005624376.3 | 0.308481 |
| GH1 | NM_001003168.1 | −0.32013 |
| GPI | XM_848765.5 | −0.00893 |
| GSK3A | XM_014116630.2 | −0.25154 |
| GSK3B | XM_535751.2 | −1.75489 |
| GZMA | XM_544335.2 | −0.00412 |
| GZMB | XM_547752.2 | −0.10001 |
| GZMH | XM_014115811.2 | 0 |
| GZMK | XM_546318.2 | −0.03601 |
| HAMP | NM_001007140.1 | −0.15904 |
| HAVCR2 | NM_001254715.1 | 0 |
| HBEGF | XM_005617277.3 | −0.15497 |
| HCK | XM_022409133.1 | 0.03551 |
| HCST | XM_850362.1 | 0.013578 |
| HDC | XM_544676.5 | 0 |
| HIF1A | NM_001287163.1 | −0.44503 |
| HLA-DRB1 | NM_001014768.1 | 1.699714 |
| HMGB1 | NM_001002937.2 | −0.55951 |
| HMGCR | XM_536323.6 | 0.329308 |
| HNF1A | XM_014107971.2 | 0.364411 |
| HRAS | XM_540523.4 | 0 |
| HSD11B1 | NM_001005756.1 | −0.13486 |
| ICAM1 | NM_001003291.1 | 0.129283 |
| ICAM2 | XM_005624303.1 | 0.062291 |
| ICAM3 | XM_003432853.4 | −0.05242 |
| ICAM4 | XM_848909.4 | 0.064752 |
| ICOS | NM_001002972.2 | 0 |
| ICOSLG | XM_014109855.2 | −0.03328 |
| IDH1 | XM_014111093.2 | 1.415037 |
| IDH2 | XM_014112432.1 | 0 |
| IDO1 | XM_532793.2 | 3.039528 |
| IDO2 | XM_022404003.1 | 0.005742 |
| IFGGC1 | NM_001313803.1 | 1.891187 |
| IFI35 | XM_548077.6 | 0.326228 |
| IFIH1 | XM_545493.4 | 0.830075 |
| IFIT2 | XM_005618758.3 | −0.32521 |
| IFNA7 | NM_001006654.1 | −0.64934 |
| IFNAR1 | XM_003640073.4 | 0.378262 |
| IFNAR2 | XM_014109800.2 | 0 |
| IFNB1 | NM_001135787.1 | −0.30662 |
| IFNG | NM_001003174.1 | 0 |
| IFNGR1 | XM_003638758.2 | 0.589601 |
| IGF1R | XM_014112396.1 | 0.117173 |
| IGF2R | NM_001122602.1 | −0.35321 |
| IGHG | IGHG1_01.1 | −0.03328 |
| IGHM | IGHM_01.1 | 0 |
| IKBKB | XM_539954.6 | 0.240094 |
| IKBKE | XM_014111236.1 | 0.329308 |
| IKBKG | XM_003640238.4 | 0 |
| IL10 | XM_850467.1 | −0.80618 |
| IL10RA | XM_005620306.1 | 0.186323 |
| IL11 | XM_022427532.1 | 0 |
| IL11RA | XM_005626742.3 | 0.296379 |
| IL12A | NM_001003293.2 | 0 |
| IL12B | NM_001003292.1 | 0 |
| IL12RB1 | XM_005632710.1 | 0.118557 |
| IL12RB2 | NM_001025399.1 | 2 |
| IL13 | NM_001003384.1 | 0 |
| IL13RA1 | XM_538150.6 | −0.27915 |
| IL13RA2 | NM_001003075.1 | 0.300395 |
| IL15 | XM_844053.1 | −0.24709 |
| IL15RA | XM_022414662.1 | 0.520073 |
| IL16 | XM_545880.4 | −0.33466 |
| IL17A | NM_001165878.1 | 0.189478 |
| IL17B | XM_022417823.1 | 1.052467 |
| IL17F | XM_538959.1 | −0.69441 |
| IL17RA | XM_543886.2 | −0.00748 |
| IL17RB | XM_022406601.1 | −0.22507 |
| IL18 | NM_001003169.1 | −0.56902 |
| IL18R1 | XM_005625993.3 | 0.245112 |
| IL18RAP | XM_538448.2 | 0.544321 |
| IL19 | XM_547384.3 | 0.060123 |
| IL1A | NM_001003157.2 | 0.238046 |
| IL1B | NM_001037971.1 | 0 |
| IL1R1 | XM_005625999.3 | −0.2475 |
| IL1R2 | XM_538451.6 | −0.16784 |
| IL1RAP | XM_014110431.1 | 0 |
| IL1RL1 | XM_005625995.3 | 0.681824 |
| IL1RL2 | XM_849121.3 | 0 |
| IL1RN | NM_001003096.1 | 0.169839 |
| IL2 | NM_001003305.1 | 0 |
| IL21 | NM_001003347.1 | −0.16008 |
| IL21R | XM_844902.4 | −0.41736 |
| IL22 | XM_538274.1 | 0 |
| IL22RA1 | XM_850020.4 | 0.263071 |
| IL22RA2 | XM_014112310.2 | 0 |
| IL23A | XM_538231.2 | 0 |
| IL23R | XM_847058.1 | −0.05242 |
| IL24 | XM_846427.3 | 0.321402 |
| IL25 | XM_005623236.3 | −0.31166 |
| IL26 | XM_846075.2 | −0.05888 |
| IL27 | XM_844736.5 | 0.262033 |
| IL29L | NM_001114853.1 | 0.101711 |
| IL2RA | NM_001003211.2 | −1.04064 |
| IL2RB | XM_847855.1 | −0.1343 |
| IL2RG | NM_001003201.1 | 0.246239 |
| IL3 | NM_001013835.1 | 0 |
| IL31RA | XM_014108431.2 | 0 |
| IL32 | XM_005621651.2 | 0.119299 |
| IL34 | XM_022419223.1 | −0.00198 |
| IL3RA | XM_014111816.1 | 0 |
| IL4 | NM_001003159.1 | −0.59704 |
| IL4R | XM_547077.2 | 0.30102 |
| IL5 | NM_001006950.1 | −0.182 |
| IL5RA | XM_022407196.1 | 0 |
| IL6 | NM_001003301.1 | −0.05242 |
| IL6R | XM_850012.1 | 0.563429 |
| IL6ST | XM_022405204.1 | 0.13993 |
| IL7 | NM_001048138.1 | 0.169839 |
| IL7R | XM_850315.1 | 0 |
| IL9 | XM_003431593.3 | 0.991067 |
| ILF3 | XM_005632891.3 | −0.25364 |
| INPPL1 | XM_542327.6 | −0.97262 |
| IRAK1 | XM_549367.6 | 0 |
| IRAK2 | XM_005632168.3 | 0 |
| IRAK4 | XM_005636956.2 | 0 |
| IRF1 | XM_003639368.4 | 0.808087 |
| IRF2 | XM_005629996.3 | 0.137264 |
| IRF3 | XM_005616307.2 | −1.11464 |
| IRF4 | XM_005640152.1 | −0.12013 |
| IRF5 | XM_014119060.2 | 0 |
| IRF8 | XM_546793.4 | −0.05242 |
| ISG15 | XM_003639053.4 | 1.146157 |
| ISG20 | XM_545847.5 | 0.237579 |
| ITGA1 | XM_005619335.3 | 0.102393 |
| ITGA2 | XM_005619334.3 | 0.215954 |
| ITGA2B | NM_001003163.2 | −0.6845 |
| ITGA4 | XM_545551.2 | 0 |
| ITGA5 | XM_022411219.1 | 0.117173 |
| ITGA6 | XM_005640324.2 | 1.451017 |
| ITGAE | XM_005624962.1 | 0.276164 |
| ITGAL | XM_547024.2 | 0.917538 |
| ITGAM | XM_014114359.1 | 0 |
| ITGAV | XM_014110784.1 | 0.344648 |
| ITGAX | XM_547049.4 | 0.124689 |
| ITGB1 | XM_022406138.1 | 1.563429 |
| ITGB2 | XM_005638976.1 | −0.24709 |
| ITGB3 | NM_001003162.1 | 0.257158 |
| ITGB4 | XM_014116208.2 | 0.39927 |
| ITK | XM_546277.2 | −0.31992 |
| JAK1 | XM_536679.2 | 0.455592 |
| JAK2 | XM_541301.2 | 0.436423 |
| JAK3 | XM_022406886.1 | 0 |
| JAM3 | XM_005619543.2 | 0.13493 |
| JAML | XM_848338.1 | −0.1835 |
| KDM5C | XM_022415517.1 | −0.18885 |
| KDR | NM_001048024.1 | 0 |
| KIT | NM_001003181.1 | −0.10731 |
| KLRA1 | NM_001002986.1 | 0.002194 |
| KLRB1 | XM_005637170.1 | −0.37906 |
| KLRD1 | NM_001048035.1 | 0.406817 |
| KLRF1 | XM_849098.1 | 0.113868 |
| KLRG1 | XM_849133.1 | 0 |
| KLRK1 | XM_005637160.1 | −0.14018 |
| KMT2A | XM_005620305.1 | 0.011229 |
| KMT2D | XM_005636883.2 | 0.117173 |
| KRT14 | NM_001253741.1 | 0.052467 |
| KRT18 | NM_001346040.1 | 0.425926 |
| KRT7 | XM_005636798.2 | −0.1835 |
| KRT8 | NM_001346039.1 | 0.040313 |
| LAG3 | XM_005637438.2 | −0.03395 |
| LAIR1 | XM_541424.2 | 0.732587 |
| LAMP1 | XM_534193.6 | −0.41568 |
| LAMP2 | XM_005641765.2 | −0.24361 |
| LAP3 | XM_005618543.2 | 1.444785 |
| LBP | XM_542993.6 | 0 |
| LCK | XM_005617639.1 | −0.05539 |
| LCN2 | XM_022423769.1 | −0.18728 |
| LCP1 | XM_005633885.2 | 0 |
| LFNG | XM_547009.5 | −0.16914 |
| LGALS1 | NM_001201488.1 | 0.113868 |
| LGALS3 | NM_001197043.1 | −0.36257 |
| LIF | NM_001197073.1 | −0.12363 |
| LOC100049001 | NM_001097555.1 | 0.113368 |
| LOC100683099 | XM_014112758.2 | −0.58496 |
| LOC100683403 | XM_005637157.3 | 0.222392 |
| LOC100686511 | XM_005616249.2 | −0.14018 |
| LOC100856270 | XM_003640212.3 | −0.152 |
| LOC102153988 | XR_293946.2 | −1.09954 |
| LOC102154078 | XR_293948.2 | 0.63743 |
| LOC102155900 | XR_293775.3 | −1.09954 |
| LOC102156614 | XM_005615781.2 | 0.261462 |
| LOC102156626 | XM_022420859.1 | 0.44887 |
| LOC102156778 | XR_002615925.1 | −1.79141 |
| LOC102156836 | XM_022404387.1 | 0.283979 |
| LOC106557449 | XM_005628291.3 | 0.334867 |
| LOC111094769 | XM_022415962.1 | −0.46042 |
| LOC475935 | XM_533144.6 | 0 |
| LOC476396 | XM_005616205.2 | 0.194048 |
| LOC476900 | XM_014106246.2 | 0.219503 |
| LOC477699 | XM_534893.6 | 0.485427 |
| LOC478984 | XM_022415348.1 | −0.19002 |
| LOC480600 | NM_001253735.1 | −0.05242 |
| LOC481722 | XM_022425780.1 | −1.19265 |
| LOC483397 | XM_540516.3 | −0.10839 |
| LOC484306 | XM_014118394.2 | 0.03145 |
| LOC484343 | XM_022423013.1 | 0.649018 |
| LOC487977 | XM_005639485.2 | −1.6065 |
| LOC488947 | XM_843271.5 | 1.096862 |
| LOC490356 | XM_022421696.1 | −0.4186 |
| LOC606890 | XM_843371.4 | 0.133387 |
| LOC609023 | XM_846203.1 | 0.081162 |
| LOC612539 | XM_014117025.2 | −0.06884 |
| LRP1 | XM_538245.7 | 0.891187 |
| LTA | XM_843793.4 | 0 |
| LTB | NM_001033510.1 | 0 |
| LTBR | XM_005637256.3 | −0.14756 |
| LTF | NM_001287076.1 | 1.222392 |
| LTK | XM_022412515.1 | 0.451755 |
| LY86 | XM_535877.7 | 0 |
| LY9 | XM_005640882.3 | 0 |
| LY96 | XM_005638051.3 | −0.11641 |
| LYN | XM_535078.6 | −0.05242 |
| MAF | XM_014113996.1 | −0.3846 |
| MAP2 | XM_022415214.1 | 0 |
| MAP2K1 | NM_001048094.1 | 0.138624 |
| MAP2K2 | NM_001048136.1 | 0.158033 |
| MAP2K4 | XM_022418586.1 | 0.441078 |
| MAP3K1 | XM_005617402.2 | 0.302484 |
| MAP3K5 | XM_533420.7 | −0.13053 |
| MAP3K7 | XM_005627639.3 | −0.3355 |
| MAP4K2 | XM_005631510.2 | −0.26946 |
| MAPK1 | NM_001110800.1 | 0.052467 |
| MAPK11 | XM_005625711.3 | 0.21579 |
| MAPK14 | NM_001003206.1 | −1.15612 |
| MAPK3 | NM_001252035.1 | 0.208179 |
| MAPK8 | XM_005637467.3 | 1.688056 |
| MAPKAPK2 | XM_014115060.2 | −0.13409 |
| MARCO | XM_005631917.3 | −0.26298 |
| MASP1 | XM_005639816.3 | −0.63387 |
| MASP2 | XM_544572.7 | −0.14018 |
| MAVS | NM_001122609.1 | −0.05242 |
| MBL2 | XM_005619088.3 | 0.171766 |
| MCAM | XM_022418207.1 | 0.289507 |
| MDM4 | XM_022415421.1 | 0.250407 |
| MEF2C | XM_014112231.2 | 0 |
| MEFV | XM_005621638.2 | −0.26298 |
| MERTK | XM_005630437.3 | 0 |
| MET | NM_001002963.1 | 0 |
| MGMT | NM_001003376.1 | −0.14581 |
| MIF | ENSCAFT00030041867.1 | −0.8834 |
| MKI67 | XM_014108788.1 | 0 |
| MME | NM_001289066.1 | −0.233 |
| MMP1 | XM_546546.2 | 3.392317 |
| MMP9 | NM_001003219.2 | 0.323774 |
| MR1 | XM_022421686.1 | −0.03699 |
| MRC1 | XM_005617091.3 | −0.36257 |
| MS4A1 | NM_001048028.1 | 0 |
| MS4A2 | NM_001003172.2 | −0.05242 |
| MSR1 | XM_843168.1 | −0.15107 |
| MST1R | XM_533823.2 | 0.107819 |
| MTOR | XM_535407.2 | −0.02168 |
| MUC1 | NM_001194977.1 | 0 |
| MX1 | NM_001003134.1 | 0.451755 |
| MYC | NM_001003246.2 | 0.072268 |
| MYD88 | XM_534223.6 | 0.265461 |
| NCAM1 | NM_001010950.1 | −0.04064 |
| NCF4 | XM_022424475.1 | −0.36494 |
| NCR1 | XM_849055.1 | 0 |
| NCR3 | XM_843835.5 | −0.38379 |
| NDRG1 | NM_001284434.1 | 0.926937 |
| NF1 | NM_001372007.1 | −0.1712 |
| NFATC1 | XM_541045.2 | 0.049464 |
| NFATC2 | XM_005635184.1 | 0.011825 |
| NFATC3 | XM_536809.2 | 0.128 |
| NFATC4 | XM_005623276.3 | −0.04064 |
| NFKB1 | NM_001003344.1 | −0.17681 |
| NFKB2 | XM_005637671.3 | 1.347923 |
| NFKBIA | XM_537413.6 | 0.501226 |
| NINJ2 | XM_849609.3 | 0.039528 |
| NKG7 | XM_005616240.2 | 0.774933 |
| NLRC5 | XM_014109333.1 | 1.289507 |
| NLRP3 | XM_843284.4 | 0.351126 |
| NOD1 | XM_005628676.3 | 1.222392 |
| NOD2 | NM_001287039.1 | −0.31677 |
| NOS2 | NM_001313848.1 | 1.084889 |
| NOTCH1 | XM_005625433.1 | 0 |
| NPNT | XM_005639244.3 | −0.77761 |
| NR1H3 | XM_022405466.1 | −0.26946 |
| NRAS | NM_001287065.1 | 0.453005 |
| NRP1 | XM_003433694.2 | 0 |
| NT5E | XM_532221.2 | 0.052467 |
| NTRK1 | XM_547525.5 | 0 |
| NUP62 | XM_014118761.2 | 0.885357 |
| OAS2 | NM_001048134.1 | 0.084037 |
| OAS3 | NM_001048091.1 | 0.460693 |
| OLIG2 | XM_005638837.3 | −1.08504 |
| OSM | XM_022410165.1 | −0.2638 |
| PALB2 | XM_845578.5 | −0.33451 |
| PARP1 | XM_022421711.1 | −0.10959 |
| PAX5 | XM_849748.1 | 0.45468 |
| PCNA | XM_534355.2 | 0.881356 |
| PDCD1 | NM_001314097.1 | −0.22792 |
| PDCD1LG2 | XM_847012.5 | 0.003691 |
| PDGFC | XM_022403541.1 | 0.943533 |
| PDGFRA | XM_005628196.2 | 0 |
| PDGFRB | NM_001003382.1 | −0.15612 |
| PDPN | NM_001003220.1 | 0 |
| PECAM1 | XM_848326.1 | 0 |
| PGR | NM_001003074.1 | −0.431 |
| PIK3C2B | XM_014111226.2 | 0.090882 |
| PIK3C2G | XM_014108746.2 | 0.203208 |
| PIK3CA | XM_545208.2 | −0.08504 |
| PIK3CD | XM_546764.6 | 0 |
| PIK3CG | XM_005630959.2 | 0 |
| PIK3R1 | XM_845248.4 | −0.25565 |
| PIN1 | XM_542080.5 | 0.772079 |
| PIP | XM_845994.5 | −0.97025 |
| PLA2G1B | NM_001003320.1 | 0 |
| PLAU | NM_001194952.1 | 0.917538 |
| PLAUR | XM_014119989.2 | 0.204471 |
| PLCG1 | XM_005635060.3 | −0.22923 |
| PNMA1 | XM_547896.6 | 0.454566 |
| PNOC | XM_543221.6 | 0.207746 |
| POU2AF1 | XM_005619799.1 | 0.194912 |
| POU2F2 | XM_014120097.1 | −1 |
| PPARG | NM_001024632.2 | 0.774933 |
| PPARGC1A | XM_014112658.2 | −0.05242 |
| PPBP | NM_001171772.2 | 0 |
| PRDM1 | XM_539068.2 | 1.392317 |
| PRF1 | XM_005618879.1 | 0 |
| PRKCD | NM_001008716.1 | 0.293476 |
| PRKCE | XM_022424679.1 | −0.4869 |
| PSMB10 | XM_546869.6 | 0.053265 |
| PSMB8 | NM_001048085.1 | 1.959358 |
| PSMB9 | NM_001048086.1 | −0.48286 |
| PTEN | NM_001003192.1 | 0 |
| PTGDR2 | NM_001048107.1 | 0 |
| PTGER2 | NM_001003170.1 | 0.003466 |
| PTGER4 | NM_001003054.1 | −0.79411 |
| PTGS2 | NM_001003354.1 | 0.465949 |
| PTHLH | NM_001003303.1 | −0.182 |
| PTK2 | XM_014118699.2 | −0.56287 |
| PTPRC | XM_005622278.1 | 0 |
| PVR | XM_005616467.3 | 0.066844 |
| PYCARD | XM_014114362.2 | 0.63743 |
| RAD51 | NM_001003043.1 | 0 |
| RAF1 | XM_005632141.1 | 0 |
| RAG2 | XM_005631105.1 | −0.17681 |
| RB1 | XM_534118.2 | 0 |
| REL | XM_005626131.1 | 0 |
| RELA | XM_005631473.2 | 0.204471 |
| RELB | XM_005616459.2 | 0 |
| RET | NM_001197099.1 | 0.124689 |
| RIPK2 | XM_005638102.3 | −0.16993 |
| RORA | XM_535503.7 | 0.072008 |
| RORC | XM_005630826.3 | 0.713379 |
| RPS6 | NM_001252170.1 | 0.662965 |
| RUNX1 | XM_844282.4 | 0.104843 |
| S100A10 | XM_003432257.4 | 0.210218 |
| S100A12 | XM_003434905.4 | 0.807355 |
| S100A4 | NM_001003161.1 | 1.038528 |
| S100A8 | NM_001146144.1 | 1.530515 |
| S100A9 | XM_005622827.2 | 0.247483 |
| S100B | XM_022413341.1 | 0.13993 |
| SAA1 | NM_001003050.1 | −0.18885 |
| SDC4 | XM_543017.4 | 0.142244 |
| SELE | NM_001003310.2 | −0.06711 |
| SELL | XM_537201.5 | −0.93289 |
| SELPLG | NM_001242719.1 | −0.21536 |
| SERPINB2 | XM_014106918.1 | 0 |
| SERPING1 | XM_022405112.1 | 0 |
| SETD2 | XM_014121990.2 | 0.117173 |
| SH2D1A | XM_847029.5 | −1.07306 |
| SHMT2 | XM_005625536.1 | 0 |
| SIGIRR | XM_022405107.1 | 0.396422 |
| SIGLEC1 | XM_542917.6 | 0.63743 |
| SLAMF1 | NM_001003084.1 | 0 |
| SLAMF6 | XM_005640886.2 | −0.65518 |
| SLAMF7 | XM_847365.1 | 0 |
| SLC11A1 | NM_001013851.1 | −0.1835 |
| SLC16A1 | XM_022405026.1 | 0.363748 |
| SLC16A3 | XM_014116216.2 | −0.17616 |
| SMAD2 | XM_022421405.1 | −0.48995 |
| SMAD3 | NM_001170829.2 | −0.10387 |
| SMARCA4 | XM_014122046.2 | 0 |
| SOCS1 | XM_005622079.1 | 0.464593 |
| SOX10 | XM_538379.6 | −0.72514 |
| SPIB | XM_003432720.4 | −0.1835 |
| SPP1 | XM_003434023.4 | 0.228624 |
| SPRY2 | XM_003433059.4 | 0 |
| SRC | XM_005635039.3 | 0 |
| ST6GAL1 | XM_005639812.2 | 0 |
| STAT1 | XM_843260.1 | 0 |
| STAT2 | XM_014117155.1 | 1.688056 |
| STAT3 | XM_548090.2 | 0 |
| STAT4 | XM_005640471.1 | −0.26298 |
| STAT5A | XM_548091.6 | 0.242292 |
| STAT5B | XM_548092.2 | −0.02554 |
| STAT6 | XM_005625532.1 | −0.24709 |
| STK11 | XM_005633151.3 | 0.218008 |
| SYK | XM_005615953.2 | 0 |
| TAL1 | XM_022427965.1 | 0 |
| TANK | XM_005640244.3 | −0.99644 |
| TAP1 | NM_001284496.2 | 0.63743 |
| TAPBP | NM_001048101.2 | −0.1835 |
| TBK1 | XM_022424157.1 | 0 |
| TBX21 | XM_548164.2 | −0.05164 |
| TCF7 | XM_003639372.2 | 0 |
| TERT | NM_001031630.1 | 0.037968 |
| TFE3 | XM_014111858.2 | −0.22827 |
| TFEB | XM_014118431.2 | −1.45003 |
| TGFB1 | NM_001003309.1 | 0 |
| TGFB2 | XM_545713.6 | −0.3202 |
| THBD | NM_001006953.2 | −0.56743 |
| THBS1 | XM_544610.2 | 0.29758 |
| THY1 | NM_001287129.1 | −0.05445 |
| TICAM1 | XM_005633023.3 | 0.315502 |
| TICAM2 | NM_001204337.1 | 1.096862 |
| TIGIT | XM_545108.4 | 0 |
| TIRAP | XM_014113300.2 | 0.117586 |
| TLR1 | XM_014112326.2 | 0.63743 |
| TLR10 | NM_001173127.1 | −0.21818 |
| TLR2 | NM_001005264.2 | 4.91E−16 |
| TLR3 | XM_005629968.3 | −0.03328 |
| TLR4 | NM_001002950.1 | −0.74764 |
| TLR5 | XM_005640757.2 | 0 |
| TLR6 | XM_022417244.1 | −0.3202 |
| TLR7 | XM_005641003.3 | 0 |
| TLR8 | XM_005641119.3 | −0.26298 |
| TLR9 | NM_001002998.1 | −0.16326 |
| TNF | NM_001003244.4 | 0.034089 |
| TNFAIP3 | XM_014112321.1 | 1.052467 |
| TNFRSF11A | XM_005615669.3 | −0.31835 |
| TNFRSF11B | XM_003639448.4 | −0.11464 |
| TNFRSF12A | NM_001193299.2 | 1.038528 |
| TNFRSF13B | XM_005620179.3 | −0.56902 |
| TNFRSF13C | XM_843968.4 | 0 |
| TNFRSF14 | XM_549666.2 | 0.152003 |
| TNFRSF17 | XM_005621530.2 | 0.429322 |
| TNFRSF18 | NM_001190744.1 | −0.13124 |
| TNFRSF1A | XM_005637258.1 | −0.02272 |
| TNFRSF1B | XM_005617982.1 | −0.44922 |
| TNFRSF4 | XM_546720.2 | −0.97728 |
| TNFRSF8 | XM_014111450.1 | 0 |
| TNFRSF9 | XM_845243.1 | 0 |
| TNFSF10 | NM_001130836.1 | 0.169839 |
| TNFSF11 | XM_846672.1 | 0.258918 |
| TNFSF12 | NM_001205168.1 | 0.013578 |
| TNFSF13 | NM_001205169.1 | −0.26298 |
| TNFSF13B | NM_001161710.2 | 0.544321 |
| TNFSF14 | XM_849235.1 | −0.2292 |
| TNFSF15 | XM_848586.3 | 0.222392 |
| TNFSF18 | XM_005622966.1 | 0 |
| TNFSF4 | XM_003639167.2 | 1.096862 |
| TNFSF8 | XM_850342.4 | 0.762961 |
| TNKS | XM_844295.5 | −0.40901 |
| TNKS2 | XM_022412163.1 | 0.277888 |
| TOLLIP | XM_540778.5 | −0.64203 |
| TOX | XM_005638006.1 | −0.13124 |
| TP53 | NM_001003210.1 | 0.243151 |
| TP63 | XM_022414176.1 | 0 |
| TRAC | Canis_TRAC.1 | 0.659031 |
| TRAF1 | XM_850435.1 | 0.141705 |
| TRAF2 | XM_005625072.2 | −0.15107 |
| TRAF3 | XM_022422397.1 | 1.321928 |
| TRAF6 | XM_003432322.4 | 0.530515 |
| TRAT1 | XM_535733.5 | −0.31521 |
| TRBC | Canis_TRBC.1 | −0.23103 |
| TREM1 | NM_001284492.1 | 0.576029 |
| TREM2 | XM_005627313.3 | 0.309855 |
| TRGC2 | Canis_TRGC2.1 | 0.78286 |
| TRGC3 | Canis_TRGC3.1 | −0.13486 |
| TRGC8 | Canis_TRGC8.1 | 0 |
| TRIB1 | NM_001313808.1 | 0.542273 |
| TSC2 | XM_859874.1 | 0.439491 |
| TUBB3 | XM_005620536.3 | 0.170553 |
| TXK | XM_022426965.1 | 0 |
| TXNIP | XM_533037.5 | 0.353976 |
| TYK2 | XM_022407058.1 | 0.16759 |
| TYMS | NM_001252174.1 | 0 |
| TYROBP | NM_001197117.1 | 0.213931 |
| VCAM1 | XM_005621302.3 | 0.222392 |
| VEGFA | NM_001003175.2 | −0.20623 |
| VEGFC | XM_540047.6 | 0 |
| VIM | NM_001287023.1 | 1.073529 |
| VSIR | XM_014112877.2 | 1.117423 |
| XCR1 | XM_005632632.3 | −0.23329 |
| ZAP70 | XM_005626046.2 | 0.488328 |
| ZEB1 | XM_003638879.4 | 0 |

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Non-Invasive GI Transcriptome Analysis and Diagnostic Platform Technology

These studies used Nanostring transcriptome quantification technology to assess the relative abundance of mRNA encoding immune-related genes (n=750 genes) directly in fecal samples from dogs (n=8) with clinically confirmed inflammatory bowel disease (IBD) and n=8 healthy, age-matched control dogs. This unbiased analysis was able to identify a panel of n=25 most highly upregulated and n=25 most highly downregulated genes in dogs with IBD compared to healthy dogs. Similar analysis can also identify significantly upregulated and downregulated genes in fecal samples from humans with IBD, as well as other companion animal species (eg, cats).

This work is important because it demonstrates the unexpected finding that mRNA is present in feces in concentrations sufficient for direct analysis by Nanostring technology. The studies also demonstrate the application of the invention to the elucidation of immune pathways in IBD, using a “whole gut analysis” approach, which allows sampling of the entire gut transcriptome throughout the GI tract, without requiring invasive GI biopsies, or amplification of mRNA by conventional RT-PCR approaches. This work also demonstrates that a similar approach can be applied to analysis of the immune or other transcriptomes in other body fluids or mucosal samples, including sputum, oral swabs, vaginal or urethral swabs, nasal swabs, ocular swabs, aural swabs, and skin swabs.

Technology overview and applications: The invention described here consists of the application of specific mammalian gene expression signatures, typically 100-200 genes, to identify and characterize specific GI diseases, in humans and veterinary species, using stool samples collective non-invasively. The test is done by using transcriptomic analysis of shed gastrointestinal cells in small stool samples to identify unique patterns of gene expression that can be used to diagnose specific GI diseases, including inflammatory bowel diseases (e.g., Crohn's disease and ulcerative colitis), functional GI diseases (e.g., irritable bowel syndrome) chronic GI infections and parasitism (e.g., Clostridium difficile, hookworms), and GI cancers (e.g., colon and gastric cancers).

The key aspects supporting this technology include the non-invasive use of stool samples, the application of next generation sequencing approaches to analyze small numbers of shed cells (both GI cells and immune and cancer cells) in stool samples, and in the identification of unique gene signatures associated with each of these diseases. This technology therefore allows specific, rapid, and non-invasive diagnosis of many different GI diseases, and also allows repeated analysis of stool samples during treatment to help guide treatment decisions.

The new technology overcomes current challenges in diagnosing complex GI diseases by directly examining the transcriptomes of GI cells in stool samples, thereby eliminating the need for expensive GI biopsy procedures such as colonoscopy and endoscopy. Because the test examines all the GI cells shed in a stool sample, it provides a much more complete picture of GI health and disease. Moreover, because diagnosis is more accurate, the new testing technology also eliminates the need for other less specific blood tests (e.g., C-reactive protein) and stool tests (e.g., calprotectin).

Several different technology platforms can be utilized to perform the GI transcriptomic analysis, using RNA extracted from small volumes (1-2 gm) of stool sample. The stool sample, collected by the patient (humans) or pet owner (canine, feline patients) is shipped to the laboratory in a suitable RNA preservative solution (e.g., Norgen Stool Sample Collection Device). In the laboratory, the RNA is extracted using an optimized extraction protocol (FIG. 9). Next, the RNA is subjected to either to full RNA sequencing, or to targeted analysis using a custom gene panel and a robust RNA analysis technology such as Nanostring. The decision on which approach to choose is based on whether an unknown disease discovery test was intended, in which case full RNA seq would be preferred, or whether the patient was being evaluated for a more specific disease condition (e.g., IBD) in which case an IBD specific diagnostic panel can be employed (e.g., Nanostring custom panel). A number of different GI specific Nanostring panels are developed, including specific diagnostic panels for IBD, GI infection, GI maldigestion/malabsorption, or GI cancer.

Finally, this GI transcriptome analysis technology also allows direct comparisons of the GI cellular transcriptome with the results of GI microbiome analysis. This comparison can allow the user (e.g., researcher, physician, veterinarian) to identify correlations between the microbiome and specific GI abnormalities, as reflected in the GI transcriptome analysis. For example, the analysis can include comparison of microbiome populations with specific GI immune cell populations, to determine directly how the two populations interact. The net result of GI transcriptome analysis can be a much richer picture of overall GI health, and a better understanding of how the microbiome impacts the GI health profile. Such a test could eventually be marketed to the general public to complement the current microbiome analysis technologies being used now being used.

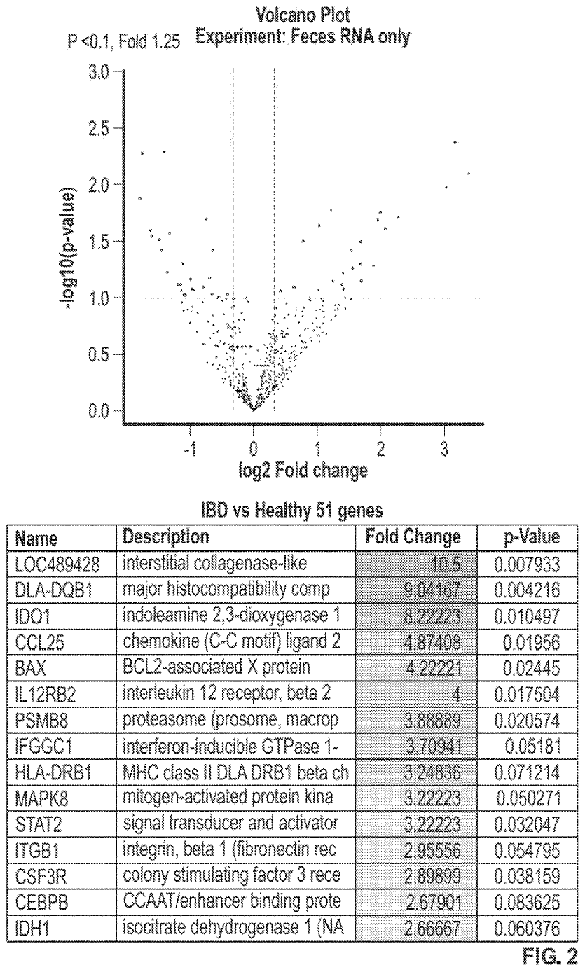

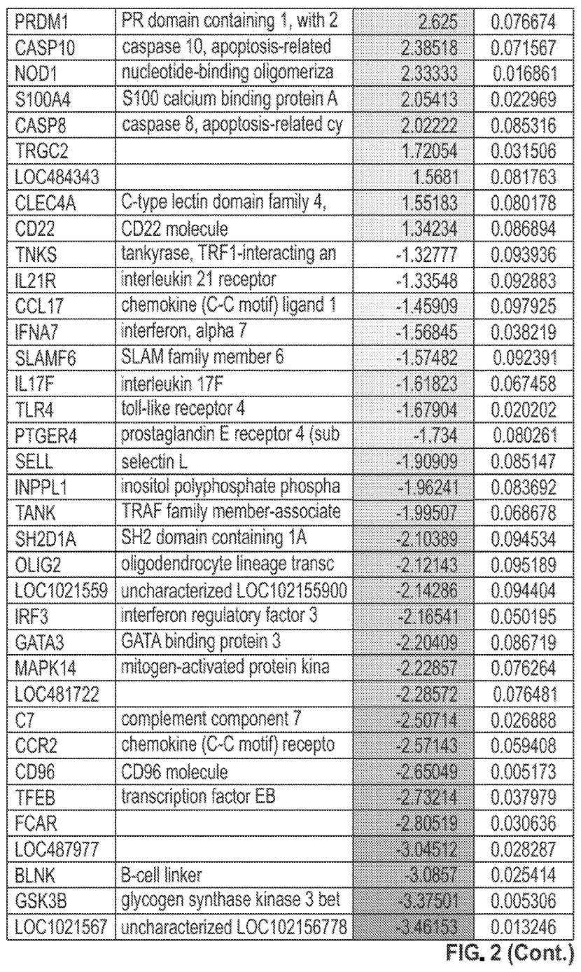

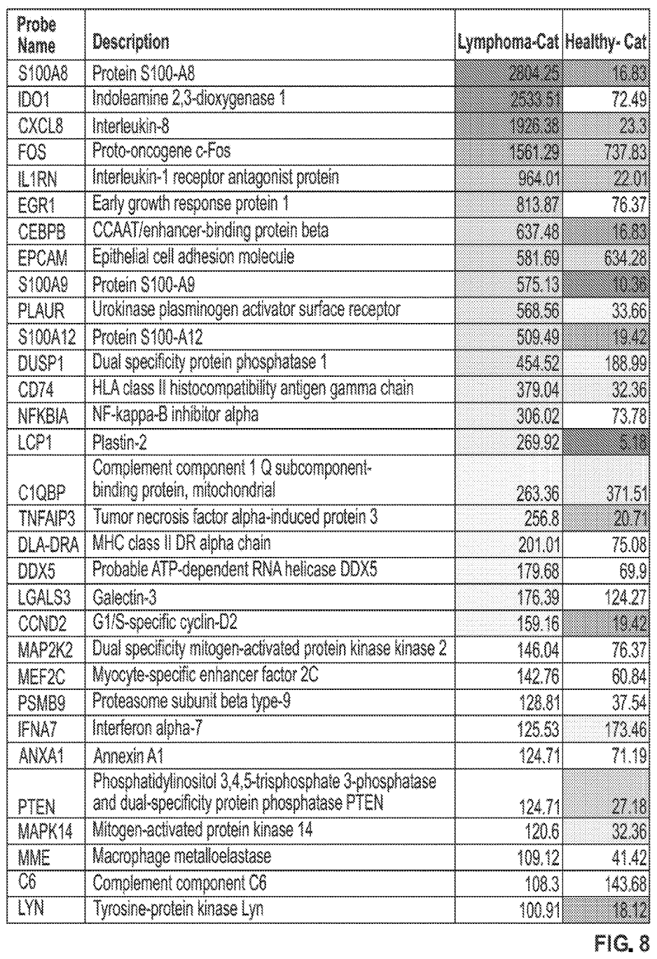

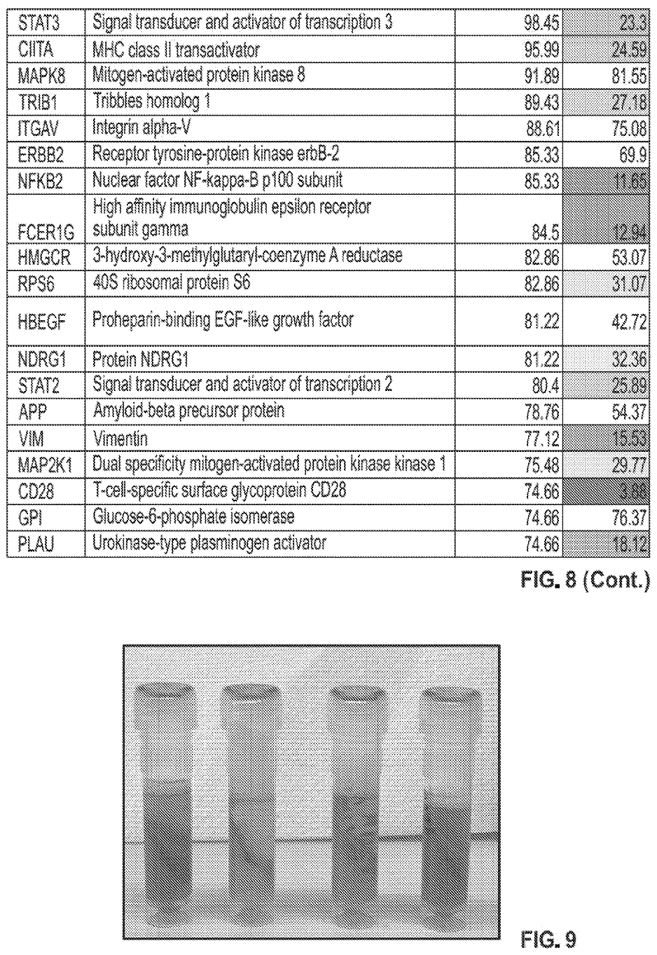

FIG. 2 shows transcriptomic analysis of immune gene expression in stool (fecal) samples from dogs with inflammatory bowel disease compared to healthy dogs. RNA was extracted from stool samples of n=8 healthy dogs and n=8 dogs with IBD using an optimized protocol (see FIG. 8), and the mRNA abundance of specific immune related genes was then quantitated, using Nanostring analysis. This study used the canine Nanostring IO panel, which consists of a canine specific immune panel of 800 immune related genes. The analysis was done using a Nanostring nCounter analysis instrument. The overall abundance of significantly upregulated and downregulated genes (p<0.1, fold change >1.25), as determined by ANOVA with adjustment for multiple comparisons) is displayed by volcano plot (left). In the heat map (right), differential gene expression analysis (DEG) was done to identify the most significantly downregulated genes (red) and upregulated genes (green). This analysis revealed that there were 51 genes whose expression was significantly different in dogs with inflammatory bowel disease compared to healthy dogs. This DEG panel can then be used to identify a unique expression signature that can be used as a specific diagnostic test for IBD in dogs. The same approach can be applied to generating a specific gene expression panel for diagnosis of IBD in humans, using only stool samples, and not requiring GI biopsies.

The data presented in FIG. 2 illustrate the ability of the non-invasive stool transcriptome analysis technology to measure eukaryotic gene expression using small stool samples, and importantly to then identify unique gene signatures associated in this case with inflammatory bowel disease. The same approach can be used to identify unique gene expression signatures associated with other GI diseases in humans and veterinary patients (dogs, cats, horses, cattle) in addition to IBD, including chronic infections, GI malabsorption or maldigestion, and GI cancers.

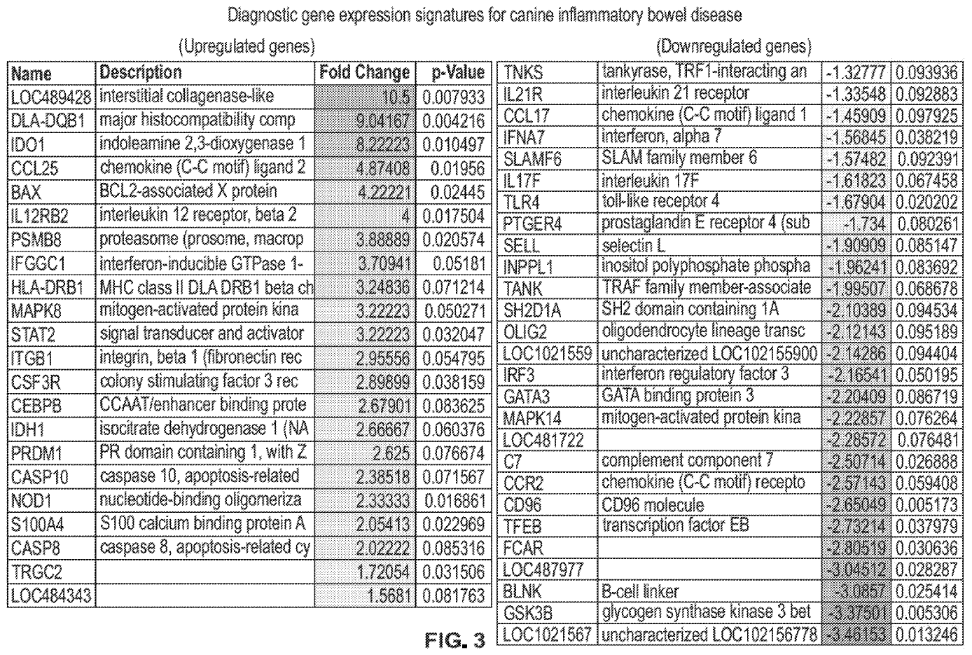

Using the stool transcriptomic analysis approach detailed in FIG. 2, a diagnostic expression signature for IBD in dogs was created (FIG. 3), consisting of 51 differentially expressed (up or down-regulated) genes. A similar unique signature, generated by analysis of stool samples from known IBD disease patients, can be used to create a diagnostic panel for screening stool specimens from human patients to identify those with a particular type of IBD, for example whether Crohn's or ulcerative colitis.

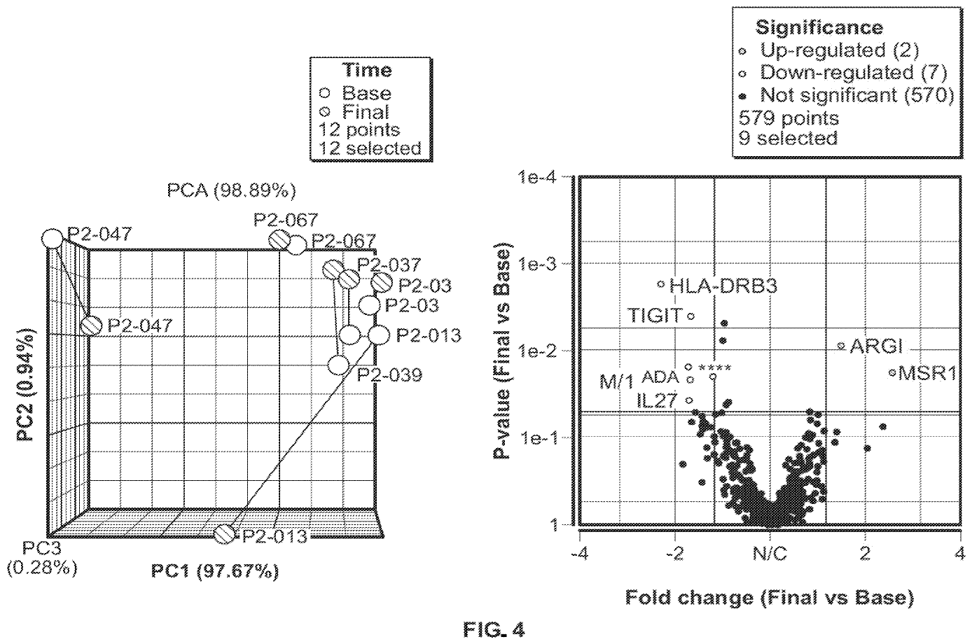

Healthy human volunteers (n=6) were placed on a dietary supplement, and changes in their GI immune gene expression were assessed using mRNA extracted from stool samples and subjected to Nanostring immune gene analysis (FIG. 4). The baseline gene expression (blue) is compared to gene expression after 8 weeks of dietary supplementation (red). Using this technology, significant changes in expression of 15 genes were identified, thereby illustrating the potential for the technology to measure changes in the GI immune transcriptome directly, bypassing the need for biopsy procedures or indirect, insensitive blood tests.

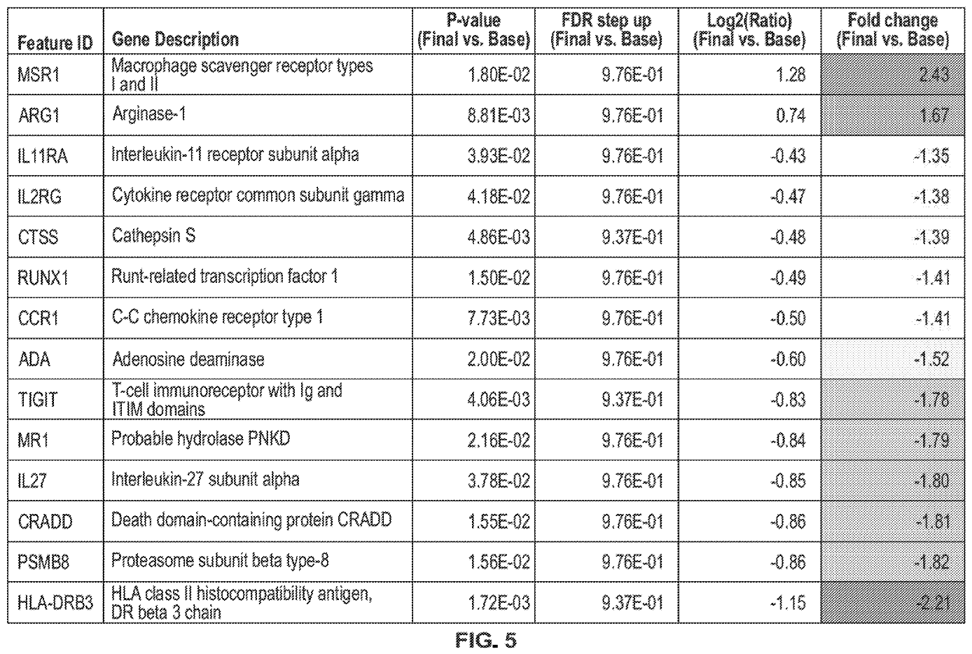

The heat map in FIG. 5 displays the 15 significantly (p<0.05) upregulated (red) or downregulated (blue) genes following a dietary trial in healthy human volunteers, as described in FIG. 4. This gene signature provides an example of the sensitivity of the non-invasive GI transcriptome technology to identify immune gene changes directly in stool samples, in a setting where conventional blood immune testing does not identify changes.

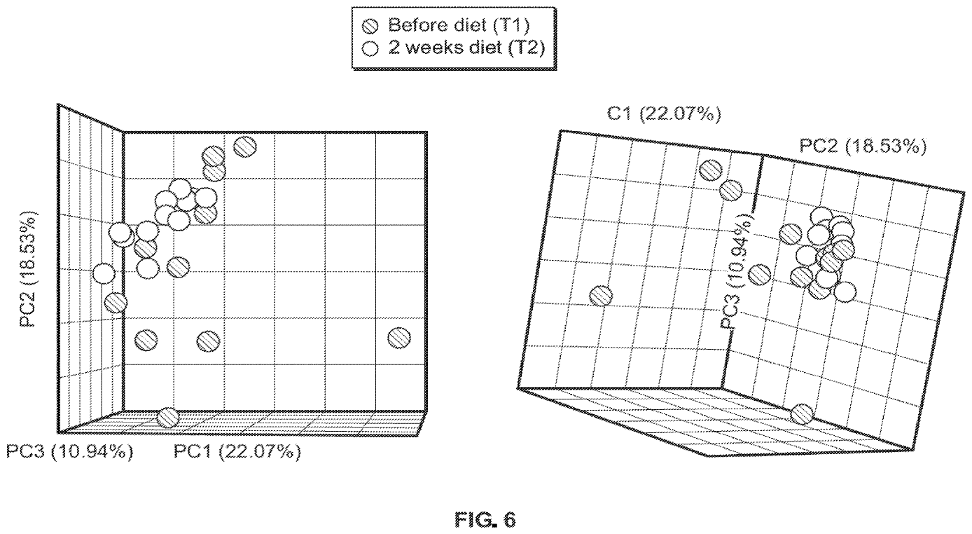

Dogs (n=12) with previously diagnosed IBD were placed on a new therapeutic diet, and stool samples collected before the diet change (magenta) and 2 weeks after the diet change (teal) (FIG. 6). RNA was extracted from the stool samples and analyzed by Nanostring analysis, using a canine 800 gene IO panel, to assess changes in the GI immune transcriptome. This analysis revealed changes in overall gene expression patterns, as assessed by principle component analysis (PCA plots).

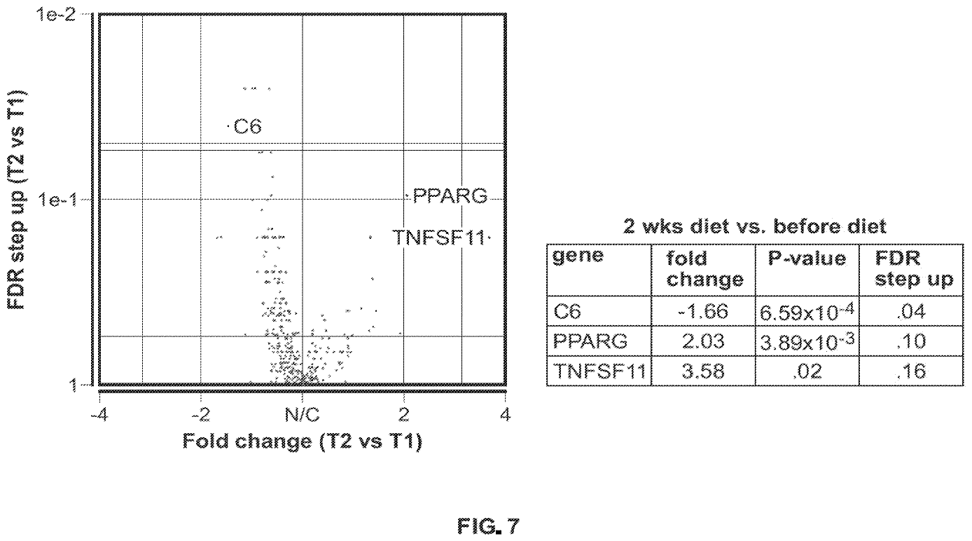

Dogs with IBD (n=12) were placed on a new elemental diet, and immune gene expression patterns were assessed using stool samples obtained before and 2-weeks after the dietary change, as described in FIG. 6. This analysis revealed 11 genes whose expression was significantly up or downregulated following the dietary change, including the 3 genes as depicted (FIG. 7). This analysis demonstrated the ability of the new stool transcriptome test to identify rapid changes in the GI immune transcriptome, using non-invasive testing with stool samples. Many of the genes identified in the dogs and cats can also be picked up in human IBD samples.

Stool samples were collected from a health cat and a cat with biopsy confirmed GI lymphoma, and immune transcriptomes were analyzed using a canine 800 gene Nanostring IO immune panel. This analysis identified 50 genes that were differentially expressed (upregulated) in the stool specimen from the cat with GI lymphoma, compared to the healthy cat (FIG. 8). These findings highlight the utility of the GI transcriptome analysis technology for use in the non-invasive diagnosis of GI cancers, including lymphoma and other types of GI neoplasia. The technology also allows the discrimination of GI inflammatory disease from cancer, with greater sensitivity than current GI biopsy procedures and analysis.

### Study Materials and Methods

Fecal samples and RNA extraction. Fecal samples (0.25 to 0.5 gms, frozen or refrigerated samples) were used for RNA analysis. Total RNA was extracted using an RNAeasy PowerSoil Total RNA kit, according to manufacturer recommendations. RNA amounts were quantitated to assure that at least 400 ng RNA was available for analysis.

Transcriptome analysis. mRNA relative abundance was determined using a Nanostring Canine IO gene panel, containing primers for 750 canine immune genes. Similar Nanostring panels exist currently for human immune genes, and custom panels can be readily created once broad screens identify specific genes of interest. For example, custom GI panels can use approximately 100-150 genes of interest for diagnostic evaluation of fecal samples (human or veterinary) as an initial screen for the presence of IBD or other GI disorders (eg, enteric infections, GI bacterial overgrowth, GI cancer). Once a diagnosis of IBD is made, more specific panels can be used to subclassify the disease into Crohn's or ulcerative colitis categories (human IBD).

The analysis of the most highly upregulated and downregulated genes in fecal samples was done using software (nCounter and Rosalind) provided by Nanostring. This analysis identified genes upregulated by at least 1.5 log 2 analysis, with a p-value set at 0.1. With additional samples (both healthy and IBD), the analysis parameters can be tightened to, for example, p=0.05 for additional statistical predictive power.

### Study Findings, Applications of the Technology

Immune gene signatures of IBD. The analysis revealed key upregulated genes useful for diagnosis of IBD in dogs (and by extension, human IBD). The key top 5 upregulated genes include MMP-1, MHC class II, IDO, CCL25, BAX, and IL12RB2. For most downregulated, the top 5 would include LOC1021567, glycogen kinase synthase 3, LOC487977, FACR, and TFEB.

Additional applications of direct transcriptome analysis using feces and other mucosal samples. The immune transcriptomic profile created using Nanostring analysis of the fecal microbiome could also be combined with microbiome sequencing to help better understand the relationship between the immunome and the microbiome in the GI tract and other sites colonized by bacteria (e.g., nasal and oral microbiomes, female reproductive tract, urinary tract, skin). This combined “immuno-microbiome” signature could be a powerful tool for disease characterization, discovery, and diagnosis in various body sites.

### Example 2. Optimized Protocol for RNA Extraction from Stool Samples for Transcriptome Analysis

### 1. Sample Collection and Handling.

Stool samples (1 to 2 gm) should be collected as quickly as possible following passage, using a stool sample collection device and then placed in a commercial RNA preservative solution (eg, Norgen Biotek, Stool Nucleic Acid Collection and Preservation Tubes) prior to shipment to the laboratory for processing. Once in RNA preservative solution, stool samples are shipped to the laboratory on a cold pack for processing.

2. Material Required for RNA Extraction from Stool Specimens.
a. RNA extraction kit. RNA extractions are performed using the Qiagen PowerMicrobiome kit, with specific additional equipment.
b. Equipment needed for extraction, not provided in kit
   Microcentrifuge (13,000×g)
   Pipettor (1.5-1000 μl)
   Vortex-Genie® 2 Vortex
   Vortex Adapter for 24 (1.5-2.0 ml) tubes (cat. no. 13000-V1-24)
   70% ethanol
   β-mercaptoethanol (β-ME)—99.9% pure, 14 M+/−Phenol-chloroform-isoamyl alcohol (25:24:1) pH 6.7-8.0 (optional)
c. Technical notes
   Prepare Solution PM1 (from PowerMicrobiome kit) by adding 10 μl β-mercaptoethanol (β-ME) for every 990 μl Solution PM1 (a total of 1 ml for each prep though you only add 650 mL per sample).
   Heat up water bath. Solution PM1 must be warmed at 55° C. for 5-10 min prior to use.
   Shake to mix Solution PM5 before use.
   Prepare DNase I stock solution by adding 550 μl RNase-Free Water to the DNase I (RNase-free) lyophilized powder and mixing gently. Aliquot the DNase I stock enzyme in 50 μl portions and store at −30° C. to −15° C. for long term storage (but do not freeze-thaw more than 3 times). To prepare DNase I solution, thaw and combine 5 μl DNase I stock enzyme with 45 μl DNase Digestion solution per prep.

### 3. Detailed RNA Extraction Procedure

Step 1. Prepare PM1-b-ME solution (1 part b-ME to 99 parts PM1, see above), warm at 55° C. for 10 minutes before starting.
Step 2. Aliquot 0.2 to 0.25 gm stool sample into a PowerBead Tube, Glass 0.1 mm (see below).
Note: If phenol-based lysis is desired, add 100 μl phenol-chloroform-isoamyl alcohol (pH 6.5-8.0) to the PowerBead Tube before adding the sample. For the dog fecal sample, I have been adding in phenol.
Step 3. Add 650 μl Solution PM1-β-ME to the PowerBead Tube. Alternatively, you may add 650 μl PM1 and 6.5 μl 1-ME to the PowerBead Tube.
Step 4. Secure the PowerBead Tube horizontally to a Vortex Adapter (cat. no. 13000-V1-24). Orient tube caps to point toward the center of the Vortex Adapter.
Step 5. Vortex at maximum speed for 10 min. Centrifuge at 13,000×g for 1 min at room temperature (15-30° C.). Transfer the supernatant (circled in photo below) to a clean 2 ml Collection Tube (provided) with a P1000 pipette.
Note: If you add phenol-chloroform-isoamyl alcohol, remove the upper aqueous layer and transfer to a clean 2 ml Collection Tube (provided).
Note: The sample is homogenized using mechanical bead beating and a lysis buffer that protects the RNA released into the supernatant. As the sample spins, proteins and cellular debris are pelleted with the beads and the supernatant contains RNA and DNA from both mammalian and bacterial cells.
Step 6. Add 150 μl Solution IRS and vortex briefly to mix. Incubate in cold room (2-8° C.) for 5 min.
Note: Solution IRS is the Inhibitor Removal Solution which completes the IRT process and removes the contaminants from the sample that would cause problems with PCR and other downstream applications.
Step 7. Centrifuge at 13,000×g for 1 min.
Step 8. Avoiding the pellet (typically big and white), transfer 650 μl of the supernatant to a clean 2 ml Collection Tube (provided).
Note: Do not transfer more than 650 μl at this step.
Step 9. Add 650 μl of Solution PM3 and 650 μl of 70% ethanol (if you want to co-purify small RNAs, then use Solution PM4 instead of ethanol). Vortex briefly to mix, the proceed to step 9.
Note: Solution PM3 contains the binding salts for total nucleic acid purification and Solution PM4 is 100% ethanol. These solutions set up the conditions for RNA and DNA binding to the Spin Filter.
Note: To prevent small RNAs (5s RNAs, tRNAs and degraded RNAs) from co-purifying with mRNA and rRNA, use 650 μl 70% ethanol instead of Solution PM4.

To purify small RNAs, such as microRNAs and siRNAs, transfer the lysate to a larger tube to accommodate a higher volume (2.6 ml) and add an additional 650 μl 100% ethanol (user supplied) to the lysate. *
Step 10. Load 650 μl of the mixture from step 8 into an MB RNA Spin Column and centrifuge the tubes at 13,000×g for 1 min. Discard the flow-through and repeat until all the supernatant has been processed through the Spin Column (typically takes 3×).
Note: Total nucleic acids are bound to the Spin Column by passing through the membrane using centrifugation.
Step 11. Shake to mix Solution PM5. Add 650 μl Solution PM5 to the MB RNA Spin Column and centrifuge at 13,000×g for 1 min.
Note: Solution PM5 is a wash buffer that contains isopropanol to remove salts from the membrane for optimal performance of the on-column DNase step.
Step 12. Discard flow-through and centrifuge at 13,000×g for 1 min to remove residual wash.
Step 13. Place the MB RNA spin column into a clean 2 ml Collection Tube (provided). To the center of the Spin Column, add 50 μl DNase I Solution (prepared by mixing 45 μl DNase Digestion Solution and 5 μl DNase I stock enzyme; see “Notes before starting”).
Step 14. Incubate at room temperature for 15 min. Add 400 μl Solution PM7 and centrifuge at 13,000×g for 1 min.
Note: DNase Digestion Solution is a DNase digestion buffer. The DNase in DNase Digestion Solution soaks into the membrane and digests the genomic DNA in the column. Solution PM7 inactivates the DNase enzyme and removes it from the column membrane along with digested DNA.
Step 15. Discard flow-through. Add 650 μl Solution PM5. Centrifuge at 13,000×g for 1 min.
Step 16. Discard flow-through. Add 650 μl Solution PM4. Centrifuge at 13,000×g for 1 min.
Note: Solution PM5 and PM4 are isopropanol- and ethanol-containing wash buffers, respectively, and are used to desalt the column before the elution step.
Step 17. Discard flow-through. Centrifuge at 13,000×g for 2 min.
Note: The final dry spin ensures all ethanol is cleared from the membrane so that the elution will be efficient.
Step 18. Place the MB RNA Spin Column into a clean 2 ml Collection Tube (provided).
Step 19. Add 100 μl RNase-Free Water (provided) to the center of the white filter membrane. Incubate at room temperature for at least 1 min.
Note: Eluting with 100 μl RNase-Free Water will maximize RNA yield. For more concentrated RNA, as little as 50 μl RNase-Free Water can be used.
Step 20. Centrifuge at 13,000×g for 1 min. Discard the MB Spin Column. The RNA is now ready for downstream applications.
Note: RNA is solubilized from the Spin Filter membrane into RNase-Free Water and is ready for use.
Step 21. Keep samples on ice and Nanodrop each sample to determine RNA concentration, then aliquot RNA samples into smaller tubes and freeze, store at −20° C.

TABLE 5
Correlation of immune transcriptome in GI tract with the selected
microbiome. A correlation (statistically significant, as defined
by FDR <0.05) between certain upregulated inflammatory immune
genes and certain families of bacteria is shown.

| | FDR step up | Pearson correlation |
|---|---|---|
| | (k_Bacteria; | (k_Bacteria; |
| | p_Fusobacteria; | p_Fusobacteria; |
| | c_Fusobacteriia; | c_Fusobacteriia; |
| Feature | o_Fusobacteriales; | o_Fusobacteriales; |
| ID | f_Fusobacteriaceae) | f_Fusobacteriaceae) |
| TNFRSF13C | 0.0192 | 0.8554 |
| CXCL10 | 0.0192 | 0.8601 |
| SHMT2 | 0.0192 | 0.8573 |
| CX3CL1 | 0.0192 | 0.8669 |
| PPARGC1A | 0.0249 | 0.8280 |
| DMBT1 | 0.0249 | 0.8403 |
| FYN | 0.0249 | 0.8259 |
| MAVS | 0.0249 | 0.8251 |
| CD163 | 0.0249 | 0.8352 |
| CCL19 | 0.0251 | 0.8216 |
| SIGIRR | 0.0293 | 0.8105 |
| CD84 | 0.0293 | 0.8105 |

| | FDR step up | Pearson correlation |
|---|---|---|
| | (k_Bacteria; | (k_Bacteria; |
| | p_Proteobacteria; | p_Proteobacteria; |
| | c_Alphaproteobacteria; | c_Alphaproteobacteria; |
| Feature | o_Rhizobiales; | o_Rhizobiales; |
| ID | f_Bradyrhizobiaceae) | f_Bradyrhizobiaceae) |
| TXNIP | 0.0042 | 0.9127 |
| | FDR step up | Pearson correlation |
| | (k_Bacteria; | (k_Bacteria; |
| | p_Proteobacteria; | p_Proteobacteria; |
| | c_Alphaproteobacteria; | c_Alphaproteobacteria; |
| Feature | o_Sphingomonadales; | o_Sphingomonadales; |
| ID | f_Sphingomonadaceae) | f_Sphingomonadaceae) |
| TXNIP | 0.0090 | 0.9006 |
| PRKCD | 0.0270 | 0.8641 |

| | FDR step up | Pearson correlation |
|---|---|---|
| | (k_Bacteria; p_Firmicutes; | (k_Bacteria; p_Firmicutes; |
| Feature | c_Clostridia; o_Clostridiales; | c_Clostridia; o_Clostridiales; |
| ID | f_Ruminococcaceae) | f_Ruminococcaceae) |
| IL17F | 0.0789 | 0.8547 |
| | FDR step up | Pearson correlation |
| | (k_Bacteria; p_Firmicutes; | (k_Bacteria; p_Firmicutes; |
| Feature | c_Clostridia; o_Clostridiales; | c_Clostridia; o_Clostridiales; |
| ID | f_Veillonellaceae) | f_Veillonellaceae) |
| NRAS | 0.0449 | 0.8684 |

| | FDR step up | Pearson correlation |
|---|---|---|
| | (k_Bacteria; | (k_Bacteria; |
| | p_Actinobacteria; | p_Actinobacteria; |
| | c_Actinobacteria; | c_Actinobacteria; |
| Feature | o_Bifidobacteriales; | o_Bifidobacteriales; |
| ID | f_Bifidobacteriaceae) | f_Bifidobacteriaceae) |
| C7 | 0.0006 | 0.9381 |
| C5 | 0.0468 | 0.8483 |
| CFD | 0.0468 | 0.8306 |
| CSF3 | 0.0468 | 0.8228 |
| LOC483397 | 0.0468 | 0.8199 |
| MAPK1 | 0.0468 | 0.8179 |

TABLE 6

| | accession | | | log2FoldChange |
|---|---|---|---|---|
| Symbol | number | Alias | Description | (IBD vs Healthy) |
| MMP1 | XM_546546.2 | MMP1\|LOC489428 | interstitial collagenase; matrix | 3.392317423 |
| | | | metallopeptidase 1 (interstitial | |
| | | | collagenase) | |
| DLA-DQB1 | NM_001014381.1 | DLA-DQB | major histocompatibility | 3.176588732 |
| | | | complex_class II_DQ beta 1 | |
| IDO1 | XM_532793.2 | INDO | indoleamine 2_3-dioxygenase 1 | 3.039528364 |
| CCL25 | NM_001005259.1 | — | chemokine (C-C motif) ligand 25 | 2.285128177 |
| BAX | NM_001003011.1 | — | BCL2-associated X protein | 2.078002512 |
| IL12RB2 | NM_001025399.1 | — | interleukin 12 receptor_beta 2 | 2 |
| PSMB8 | NM_001048085.1 | ATP6V0D2 | proteasome (prosome_macropain) | 1.959358016 |
| | | | subunit_beta type_8 | |
| | | | (large multifunctional | |
| | | | peptidase 7) | |
| STAT2 | XM_014117155.1 | — | signal transducer and activator | 1.688055994 |
| | | | of transcription 2_113 kDa | |
| CSF3R | XM_005628934.3 | — | colony stimulating factor 3 | 1.535550307 |
| | | | receptor (granulocyte) | |
| NOD1 | XM_005628676.3 | — | nucleotide-binding | 1.222392421 |
| | | | oligomerization domain | |
| | | | containing 1 | |
| S100A4 | NM_001003161.1 | MTS1 | S100 calcium binding protein A4 | 1.038528229 |
| TRGC2 | Canis_TRGC2.1 | NA | T Cell Receptor Gamma | 0.78286036 |
| | | | Constant 2 | |
| IFNA7 | NM_001006654.1 | CaIFN- | interferon_alpha 7 | −0.649341729 |
| | | alpha7 | | |
| TLR4 | NM_001002950.1 | — | toll-like receptor 4 | −0.747635312 |
| C7 | XM_022417892.1 | — | complement component 7 | −1.326044203 |
| CD96 | XM_545094.2 | — | CD96 molecule | −1.406255606 |
| TFEB | XM_014118431.2 | — | transcription factor EB | −1.450032921 |
| FCAR | XM_014116440.1 | NA | immunoglobulin alpha Fc | −1.488100961 |
| | | | receptor | |
| LOC487977 | XM_005639485.2 | NA | CD200 receptor 1; cell surface | −1.606495662 |
| | | | glycoprotein CD200 receptor 1 | |
| BLNK | XM_005637573.3 | — | B-cell linker | −1.625604485 |
| GSK3B | XM_535751.2 | — | glycogen synthase kinase 3 beta | −1.754887502 |
| LOC102156778 | XR_002615925.1 | — | uncharacterized LOC102156778, | −1.791413378 |
| | | | Predicted model non-protein- | |
| | | | coding transcript | |

### Example 3. Gastrointestinal Health Diagnostic

Described herein is gastrointestinal transcriptome analysis using stool samples for detection and classification of immune, neoplastic, and infectious diseases of the GI tract.

The Enteric-Immune Assay, or EIA, is a diagnostic test for assessing the overall immune health status of the GI tract, using only small specimens of stool for testing. The EIA assesses expression of a panel of immune genes (up to at least 750) by direct measurement of mRNA levels in feces, using NanoString methodology. The test comprises a stool collection vial with RNA preservative, 250-500 ng RNA extracted from 1-2 g of feces, a custom designed NanoString cassette capable of quantitating up to at least 750 immune genes of interest, and proprietary algorithms designed for classification of gene signatures associated with particular types of inflammatory bowel disease (e.g., ulcerative colitis, celiac disease, Crohn's disease).

By measuring a large panel of genes, the EIA diagnostic test allows much greater precision in detecting GI immune abnormalities associated with clinical or subclinical inflammatory bowel diseases. The inclusion of large numbers of immune genes in the EIA panel also provides greater insights into GI disease immune mechanism, which in turn allows for disease subclassification and more personalized medical diagnoses and treatment planning. Use of NanoString technology for mRNA quantitation is robust in that RNA quality is not a major factor in assay performance. The new EIA test developed herein allows the detection and classification of GI inflammatory diseases without the need for GI biopsy or other invasive procedures.

The EIA test is innovative in that it directly measures mRNA abundance in stool specimens, without the need for technically complicated amplification steps typically required in other tests for measurement of mRNA concentrations (e.g., RT-PCR tests). It had been assumed previously that mRNA shed from immune cells and epithelial cells in the gut would be too rapidly degraded for effective detection by conventional RT-PCR or by Nanostring approaches. However, we have discovered that in fact sufficient mRNA from host immune cells (i.e., mammalian mRNA) is present in feces to allow direct detection and quantification by NanoString analysis is possible, using simple fecal RNA extraction from small amounts of feces (e.g., 1-2 gm), without further target amplification or other laborious preparation of larger fecal samples. In addition, sufficient mRNA is preserved in frozen or refrigerated fecal samples to be readily detected.

Proof of concept data has been obtained using canine fecal samples from healthy dogs and dogs with inflammatory bowel disease (IBD), which demonstrated that the test can generate immune gene signatures that discriminated healthy from IBD animals. Importantly, many GI immune responses in human and dog IBD patients are shared between dogs and humans, thus the same EIA technology can discriminate healthy vs IBD human patients. Moreover, the fact that mammalian mRNA can be quantitated in dogs strongly indicates that the EIA test can detect host mRNA from mammalian species, as there is no reason based on numerous prior studies (e.g., histopathology, immuno-histochemistry, flow cytometry, RNAseq) to believe that the dog GI tract and immune cell populations are substantially different in number or function from other major species. Moreover, this study has found that the EIA gene signatures associated with IBD in dogs overlap considerably with human IBD-associated genes.

This test has several applications for diagnosis and management of GI disorders (inflammatory diseases, cancer, chronic infections, among others) of humans, companion animals (e.g., dogs, cats, horses) and livestock (e.g., cattle, sheep, swine, poultry). While the EIA test was developed for detection and classification of IBD In humans, numerous other possible applications exist. For example, a cancer screening test can be designed for use with stool samples for early detection of GI cancers, including colorectal cancer, gastric cancer, or esophageal cancer. A custom probe set can be readily designed for detection of transcripts associated with specifically mutated genes in CRC, including mutated KRAS and b-actin and hemoglobin genes (fecal blood). Multiple probe sets spanning common KRAS mutations can be included, as well as mutated genes associated with gastric cancer (e.g., mutant TP53, erbB-2), and built into a single GI cancer panel.

Another application of the technology is a custom stool sample test for diagnosis of chronic infections of the GI tract, including the viral pathogens CMV, herpesvirus infection, HPV infection, and the bacterial pathogens Yersinia, Mycobacterium (both M. tuberculosis and M. avium), Actinomyces, and Treponema. A diagnostic test for enteric pathogens would include probe sets specific for individual pathogens and could be designed as a narrow or broad screening test for chronic GI infections.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of measuring a level of an RNA in a stool sample, comprising:
a) obtaining a stool sample;
b) extracting the RNA from the stool sample; and
c) measuring the level of the RNA.

2. The method of claim 1, wherein the stool sample of step a) is placed or stored in an RNA preservation solution.

3. The method of claim 1, wherein step b) further comprises:
adding the stool sample in a tube that comprises a lysis buffer, wherein the lysis buffer comprises β-mercaptoethanol;
mixing the stool sample with the lysis buffer; and
centrifuging the mixed sample thereby obtaining a first supernatant portion.

4. The method of claim 3, further comprising adding a solution comprising phenol, chloroform, and isoamyl alcohol into the tube prior to adding the stool sample.

5. The method of claim 3, further comprising adding a solution comprising phenol into the tube prior to adding the stool sample.

6. The method of claim 3, further comprising:
transferring the first supernatant portion into a tube;
mixing the first supernatant portion with an inhibitor removal solution in the tube; and
centrifuging the mixed sample thereby obtaining a second supernatant portion.

7. The method of claim 6, further comprising:
transferring the second supernatant portion into a tube;
mixing the second supernatant portion with a solution comprising binding salts and ethanol; and
passing the mixture through a binding membrane thereby binding the RNA onto the membrane.

8. The method of claim 7, further comprising adding a DNase to the binding membrane.

9. The method of claim 7, further comprising washing the binding membrane with a washing buffer comprising isopropanol or ethanol.

10. The method of claim 7, further comprising adding water to the binding membrane thereby eluting the RNA in the water.

11. The method of claim 1, wherein the level of the RNA is measured by an RNA sequencing assay, a hybridization-based array, or a polymerase chain reaction (PCR)-based assay.

12. The method of claim 1, wherein the level of the RNA is measured by one of more primers or probes specific for RNAs encoded by genes selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567.

13. The method of claim 1, wherein the level of the RNA is measured by one of more primers or probes specific for RNAs encoded by genes selected from the group consisting of LOC489428, DLA-DQB1, IDO1, CCL25, BAX, IL12RB2, PSMB8, STAT2, CSF3R, NOD1, S100A4, and TRGC2.

14. The method of claim 1, wherein the level of the RNA is measured by one of more primers or probes specific for RNAs encoded by genes selected from the group consisting of IFNA7, TLR4, C7, CD96, TFEB, FCAR, LOC487977, BLNK, GSK38, and LOC1021567.

15. The method of claim 1, wherein the level of the RNA is measured by one of more primers or probes specific for RNAs encoded by genes selected from the group consisting of TNFRSF13C, CXCL10, SHMT2, CX3CL1, PPARGC1A, DMBT1, FYN, MAVS, CD163, CCL19, SIGIRR, CD84, TXNIP, TXNIP, PRKCD, IL17F, NRAS, C7, C5, CFD, CSF3, LOC483397, and MAPK1.

16. The method of claim 1, wherein the level of the RNA is measured by one of more primers or probes specific for RNAs encoded by genes selected from the group consisting of MSR1, ARG1, IL11RA, IL2RG, CTSS, RUNX1, CCR1, ADA, TIGIT, MR1, IL27, CRADD, PSMB8, and HLA-DRB3.

17. The method of claim 1, wherein the level of the RNA is measured by one of more primers or probes specific for RNAs encoded by genes selected from the group consisting of S100A8, IDO1, CXCL8, FOS, IL1RN, EGR1, CEBPB, EPCAM, S100A9, PLAUR, S100A12, DUSP1, CD74, NFKBIA, LCP1, C1QBP, TNFAIP3, DLA-DRA, DDX5, LGALS3, CCND2, MAP2K2, MEF2C, PSMB9, IFNA7, ANXA1, PTEN, MAPK14, MME, C6, LYN, STAT3, CIITA, MAPK8, TRB1, ITGAV, ERBB2, NFKB2, FCER1G, HMGCR, RPS6, HBEGF, NDRG1, STAT2, APP, VIM, MAP2K1, CD28, and GPI.

18.-33. (canceled)

34. A method for treating inflammatory bowel disease (IBD) in a human or a companion animal, comprising:
obtaining a biological sample from the human or companion animal;
quantifying an expression level of one or more genes relative to a reference control, wherein the one or more genes are selected from the group consisting of LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, CD22, TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567;
determining that the animal is susceptible to or suffering from IBD if the expression level of one or more genes selected from LOC489428, DLA-DQB1, IDO1, CCL25, BAX, IL12RB2, PSMB8, IFGGC1, HLA-DRB1, MAPK8, STAT2, ITGB1, CSF3R, CEBPB, IDH1, PRDM1, CASP10, NOD1, S100A4, CASP8, TRGC2, LOC484343, CLEC4A, and CD22 in the biological sample is higher in comparison to the reference control and/or if the expression level of one or more genes selected from TNKS, IL21R, CCL17, IFNA7, SLAMF6, IL17F, TLR4, PTGER4, SELL, INPPL1, TANK, SH2D1A, OLIG2, LOC1021559, IRF3, GATA3, MAPK14, LOC481722, C7, CCR2, CD96, TFEB, FCAR, LOC487977, BLNK, GSK3B, and LOC1021567 in the biological sample is lower in comparison to the reference control; and
if the animal is susceptible to or suffering from IBD:
i) administering to the companion animal an effective amount of a therapeutic agent for treating the IBD,
ii) changing the diet of the human or companion animal, or
iii) performing a fecal transfaunation.

35.-56. (canceled)

57. A method for treating a gastrointestinal disorder in a human or companion animal, comprising:
obtaining a stool sample from the human or companion animal;
quantifying an expression level of one or more genes relative to a reference control, wherein the one or more genes are selected from the group consisting of MSR1, ARG1, IL11RA, IL2RG, CTSS, RUNX1, CCR1, ADA, TIGIT, MR1, IL27, CRADD, PSMB8, and HLA-DRB3;
determining that the human or companion animal is susceptible to or suffering from the gastrointestinal disorder if the expression level of one or more genes selected from MSR1 and ARG1 in the biological sample is higher in comparison to the reference control and/or if the expression level of one or more genes selected from IL11RA, IL2RG, CTSS, RUNX1, CCR1, ADA, TIGIT, MR1, IL27, CRADD, PSMB8, and HLA-DRB3 in the biological sample is lower in comparison to the reference control; and
if the human or companion animal is susceptible to or suffering from the gastrointestinal disorder:
i) administering to the human or companion animal an effective amount of a therapeutic agent for treating the gastrointestinal disorder, and/or
ii) changing the diet of the human or companion animal.

58.-62. (canceled)
